SEQUENCE LISTING (220 sequences)

SEQ ID NO: 1 - DNA, 13139 bp, Artificial Sequence
  Description of Artificial Sequence Rosa26 locus sequence
aagcttctcacgtagcaaccagagctccagagccagcagctgctgccgccttgtatactcactcctgtgatccaacacaggagcaaccttttctttaccccacccccacttcttaacacacttttttttggggggggggggggaacaagtgctccatgctggaaggattggaactatgcttttagaaaggaacaatcctaaggtcacttttaaattgaggtctttgatttgaaaatcaacaaataccaaattccaaatattcgttttaattaaaccagcaatgtggatataagcattaagttttagttttaaaaaggtcaattttccaaacattcagcaatcatatttaaatttacagctaggaacaagagccttgggtcatgtcctaccaaagaacataactcaatattctacacatgacaatctgaataaccttaaagcctctaatcccataacaggccacaaattttggacagagaactaatgatcctcctgagaaaactggaagaaatccagggaaaagaaattcctgtgtcctccaaactcagaaatctctaattatgtcagtattctctgctttagtcctaggtcagattgcacacatctaaaataacctcttaaagttttcctcctagcgacctaaaccattattaatatcaaattaaccatcaaaacactttcctctcaatatgctgcacacaaacctcctcctggaacctcctccatctggatcctccccaatcaaaagtataggtatttaacatataagcaaggaagtaatgtaaacatgaccttggtcacaaatatgtcatctaaaaacaatttagtcaaggtatggaggaaattcgagaacctgaatctttttaagtattttgagcacaggaacaattggcaaaaggaatccaggtatagacaaaacccagagcccagagctctgggcgaaaaatgagttgctggtgaagacgttacacaagtaacatgagaaagcagaaaatgcaggtcatccacgcacccctgacccaggccagcagggcgggctgcagcatcagtacacaggagaaagatccttattcctaagaatgagaaaggcaaaggcgcccgatagaataaattagcatagaaggggctttcccaggagttaaaactttccttctgagcgattacctactaaaaccagggcttttgcccactaccatttacctaggatcttggcttgcacggattcataggggcatatccctccccctcttctttagagtcgttcttaaaagatcgctctccacgccctaggcagggaaaacgacaaaatctggctcaattccaggctagaaccctacaaattcaacagggatatcgcaaggatactggggcatacgccacagggagtccaagaatgtgaggtgggggtggcgaaggtaatgtctttggtgtgggaaaagcagcagccatctgagataggaactggaaaaccagaggagaggcgttcaggaagattatggaggggaggactgggcccccacgagcgaccagagttgtcacaaggccgcaagaacaggggaggtggggggctcagggacagaaaaaaaagtatgtgtattttgagagcagggttgggaggcctctcctgaaaagggtataaacgtggagtaggcaatacccaggcaaaaaggggagaccagagtagggggaggggaagagtcctgacccagggaagacattaaaaaggtagtggggtcgactagatgaaggagagcctttctctctgggcaagagcggtgcaatggtgtgtaaaggtagctgagaagacgaaaagggcaagcatcttcctgctaccaggctggggaggcccaggcccacgaccccgaggagagggaacgcagggagactgaggtgacccttctttcccccggggcccggtcgtgtggttcggtgtctcttttctgttggacccttaccttgacccaggcgctgccggggcctgggcccgggctgcggcgcacggcactcccgggaggcagcgagactcgagttaggcccaacgcggcgccacggcgtttcctggccgggaatggcccgtacccgtgaggtgggggtggggggcagaaaaggcggagcgagcccgagcggggagggggagggccaggggcggagggggccggcactactgtgttggcggactggcgggactagggctgcgtgagtctctgagcgcaggcgggcggcggccgcccctcccccggcggcggcagcggcggcagcggcggcagctcactcagcccgctgcccgagcggaaacgccactgaccgcacggggattcccagtgccggcgccaggggcacgcgggacacgccccctcccgccgcgccattggcctctccgcccaccgccccacacttattggccggtgcgccgccaatcagcggaggctgccggggccgcctaaagaagaggctgtgctttggggctccggctcctcagagagcctcggctaggtaggggatcgggactctggcgggagggcggcttggtgcgtttgcggggatgggcggccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagagtgtactataaggttgatggtaagcttataaggaacttgaaagtggagtaactactccatttctctgaggggagaattaaaatttttgaccaagtgttgttgagccactgagaatggtctcagaacataacttcttaaggaaccttcccagattgccctcaacactgcaccacatttggtcctgcttgaacattgccatggctcttaaagtcttaattaagaatattaattgtgtaattattgtttttcctcctttagatcattccttgaggacaggacagtgcttgtttaaggctatatttctgctgtctgagcagcaacaggtcttcgagatcaacatgatgttcataatcccaagatgttgccatttatgttctcagaagcaagcagaggcatgatggtcagtgacagtaatgtcactgtgttaaatgttgctatgcagtttggatttttctaatgtagtgtaggtagaacatatgtgttctgtatgaattaaactcttaagttacaccttgtataatccatgcaatgtgttatgcaattaccattttaagtattgtagctttctttgtatgtgaggataaaggtgtttgtcataaaatgttttgaacatttccccaaagttccaaattataaaaccacaacgttagaacttatttatgaacaatggttgtagtttcatgcttttaaaatgcttaattattcaattaacaccgtttgtgttataatatatataaaactgacatgtagaagtgtttgtccagaacatttcttaaatgtatactgtctttagagagtttaatatagcatgtcttttgcaacatactaacttttgtgttggtgcgagcaatattgtgtagtcattttgaaaggagtcatttcaatgagtgtcagattgttttgaatgttattgaacattttaaatgcagacttgttcgtgttttagaaagcaaaactgtcagaagctttgaactagaaattaaaaagctgaagtatttcagaagggaaataagctacttgctgtattagttgaaggaaagtgtaatagcttagaaaatttaaaaccatatagttgtcattgctgaatatctggcagatgaaaagaaatactcagtggttcttttgagcaatataacagcttgttatattaaaaattttccccacagatataaactctaatctataactcataaatgttacaaatggatgaagcttacaaatgtggcttgacttgtcactgtgcttgttttagttatgtgaaagtttggcaataaacctatgtcctaaatagtcaaactgtggaatgactttttaatctattggtttgtctagaacagttatgttgccatttgccctaatggtgaaagaaaaagtggggagtgccttggcactgttcatttgtggtgtgaaccaaagaggggggcatgcacttacacttcaaacatccttttgaaagactgacaagtttgggtcttcacagttggaattgggcatcccttttgtcagggagggagggagggagggaggctggcttgttatgctgacaagtgtgattaaattcaaactttgaggtaagttggaggaacttgtacattgttaggagtgtgacaatttggactcttaatgatttggtcatacaaaatgaacctagaccaacttctggaagatgtatataataactccatgttacattgatttcacctgactaatacttatcccttatcaattaaatacagaagatgccagccatctgggccttttaacccagaaatttagtttcaaactcctaggttagtgttctcactgagctacatcctgatctagtcctgaaaataggaccaccatcacccccaaaaaaatctcaaataagatttatgctagtgtttcaaaattttaggaataggtaagattagaaagttttaaattttgagaaatggcttctctagaaagatgtacatagtgaacactgaatggctcctaaagagcctagaaaactggtactgagcacacaggactgagaggtctttcttgaaaagcatgtattgctttacgtgggtcacagaaggcaggcaggaagaacttgggctgaaactggtgtcttaagtggctaacatcttcacaactgatgagcaagaactttatcctgatgcaaaaaccatccaaacaaactaagtgaaaggtggcaatggatcccaggctgctctagaggaggacttgacttctcatcccatcacccacaccagatagctcatagactgccaattaacaccagcttctagcctccacaggcacctgcactggtacacataatttcacacaaacacagtaagaagccttccacctggcatggtattgcttatctttagttcccaacacttgggaggcagaggccagccagggctatgtgacaaaaaccttgtctagaggagaaacttcatagcttatttcctattcacgtaaccaggttagcaaaatttaccagccagagatgaagctaacagtgtccactatatttgtagtgttttaagtcaattttttaaatatacttaatagaattaaagctatggtgaaccaagtacaaacctggtgtattaacttgagaacttagcataaaaagtagttcatttgttcagtaaatattaaatgcttactggcaaagattatgtcaggaacttggtaaatggtgatgaaacaatcatagttgtacatcttggttctgtgatcaccttggtttgaggtaaaagtggttcctttgatcaaggatggaattttaagtttatattcaatcaataatgtattattttgtgattgcaaaattgcctatctagggtataaaacctttaaaaatttcataataccagttcattctccagttactaattccaaaaagccactgactatggtgccaatgtggattctgttctcaaaggaaggattgtctgtgccctttattctaatagaaacatcacactgaaaatctaagctgaaagaagccagactttcctaaataaataactttccataaagctcaaacaaggattacttttaggaggcactgttaaggaactgataagtaatgaggttacttatataatgatagtcccacaagactatctgaggaaaaatcagtacaactcgaaaacagaacaaccagctaggcaggaataacagggctcccaagtcaggaggtctatccaacacccttttctgttgagggccccagacctacatattgtatacaaacagggaggtgggtgattttaactctcctgaggtaccttggtaaatctttgtcctgagtaagcagtacagtgtacagtttacattttcatttaaagatacattagctccctctaccccctaagactgacaggcactttgggggtggggagggctttggaaaataacgcttccatacactaaaagagaaatttctttaattaggcttgttggttccatacatctactggtgtttctactacttagtaatattataatagtcacacaagcatctttgctctgtttaggttgtatatttattttaaggcagatgataaaactgtagatcttaagggatgcttctgcttctgagatgatacaaagaatttagaccataaaacagtaggttgcacaagcaatagaatatggcctaaagtgttctgacacttagaagccaagcagtgtaggcttcttaagaaataccattacaatcaccttgctagaaatcaagcattctggagtggtcaagcagtgtaacctgtactgtaagttacttttctgctatttttctcccaaagcaagttctttatgctgatatttccagtgttaggaactacaaatattaataagttgtcttcactcttttctttaccaaggagggtctcttccttcatcttgatctgaaggatgaacaaaggcttgagcagtgcgctttagaagataaactgcagcatgaaggcccccgatgttcacccagactacatggacctttcgccacacatgtcccattccagataaggcctggcacacacaaaaaacataagtcattaggctaccagtctgattctaaaacaacctaaaatcttcccacttaaatgctatgggtggtgggttggaaagttgactcagaaaatcacttgctgtttttagagaggatctgggttcagtttctgatacattgtggcttacaactataactccagttctagggggtccatccaacatcctcttctgttgagggcaccaaataaatgtattgtgtacaaacagggaggtgagtgatttaactctcgtgtatagtaccttggtaaaacatttcttgtcctgagtaagcagtacagctctgcctgtccctggtctacagacacggctcatttcccgaaggcaagctggatagagattccaatttctcttcttggatcccatcctataaaagaaggtcaagtttaatctattgcaaaaggtaaataggtagtttcttacatgagacaagaacaaatcttaggtgtgaagcagtcatcttttacaggccagagcctctattctatgccaatgaaggaaactgttagtccagtgttatagagttagtccagtgtatagttttctatcagaacactttttttttaaacaactgcaacttagcttattgaagacaaaccacgagtagaaatctgtccaagaagcaagtgcttctcagcctacaatgtggaataggaccatgtaatggtacagtgagtgaaatgaattatggcatgtttttctgactgagaagacagtacaataaaaggtaaactcatggtatttatttaaaaagaatccaatttctacctttttccaaatggcatatctgttacaataatatccacagaagcagttctcagtgggaggttgcagatatcccactgaacagcatcaatgggcaaaccccaggttgtttttctgtggagacaaaggtaagatatttcaatatattttcccaagctaatgagatggctcagcaaataatggtactggccattaagtctcatgacctgagcttgatcctcagggaccatgtggtacaaggagagacctaaatccttcagttggacttcaatcttctaccctcatgtccacacacaaataaatacaataaaaaacattctgcagtcgaatttctaaaagggcgaat

SEQ ID NO: 2 - DNA, 5409 bp, Artificial Sequence
  Description of Artificial Sequence sequence of homology region
caggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatc

SEQ ID NO: 3 - DNA, 4413 bp, Artificial Sequence
  Description of Artificial Sequence Sequence of Fluc-hygro insert
tctaggtaaccgatatccctgcaggggtgacctgcacgtctagggcgcagtagtccagggtttccttgatgatgtcatacttatcctgtcccttttttttccacagctcgcggttgaggacaaactcttcgcggtctttccagtactcctgcaggtgactgactgagtcgagatctgcgatctaagtaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagaccggttcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgcccccgatccgtcgacgccctaaggccatagcggccgccctgaggccgcgggcgatcgcctaggggtaaccgaagttcctatactttctagagaataggaacttcggaataggaacttcttataatctagaagatctggatccacgattcgagggcccctgcaggtcaattctaccgggtaggggaggcgcttttcccaaggcagtctggagcatgcgctttagcagccccgctggcacttggcgctacacaagtggcctctggcctcgcacacattccacatccaccggtagcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcgaaggtcctcccgaggcccggcattctcgcacgcttcaaaagcgcacgtctgccgcgctgttctcctcttcctcatctccgggcctttcgacgatccagccgccaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgccatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtcgatgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcctgcaggaattcatatgaagttcctatactttctagagaataggaacttcggaataggaacttcaaaatgtcgcggcgcgccggtaaccgaagttcctatactttctagagaataggaacttcggaataggaacttcaagcttaagcgc

SEQ ID NO: 4 - DNA, 14947 bp, Artificial Sequence
  Description of Artificial Sequence Targeting vector for Rosa26 locus with a Fluc-hygro insert
ctagggataacagggtaatatagccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctaggtaaccgatatccctgcaggggtgacctgcacgtctagggcgcagtagtccagggtttccttgatgatgtcatacttatcctgtcccttttttttccacagctcgcggttgaggacaaactcttcgcggtctttccagtactcctgcaggtgactgactgagtcgagatctgcgatctaagtaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagaccggttcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgcccccgatccgtcgacgccctaaggccatagcggccgccctgaggccgcgggcgatcgcctaggggtaaccgaagttcctatactttctagagaataggaacttcggaataggaacttcttataatctagaagatctggatccacgattcgagggcccctgcaggtcaattctaccgggtaggggaggcgcttttcccaaggcagtctggagcatgcgctttagcagccccgctggcacttggcgctacacaagtggcctctggcctcgcacacattccacatccaccggtagcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcgaaggtcctcccgaggcccggcattctcgcacgcttcaaaagcgcacgtctgccgcgctgttctcctcttcctcatctccgggcctttcgacgatccagccgccaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgccatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtcgatgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcctgcaggaattcatatgaagttcctatactttctagagaataggaacttcggaataggaacttcaaaatgtcgcggcgcgccggtaaccgaagttcctatactttctagagaataggaacttcggaataggaacttcaagcttaagcgctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagcacgtgttaactcgagctgcaggattcgagggccccggcaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatgcaccgtccttgccatcatggcctcgtaccccggccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgaggggatcgatccgtcctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctaggccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgtcgagatctagatatcgatggccatagagttacg

SEQ ID NO: 5 - DNA, 4665 bp, Artificial Sequence
  Description of Artificial Sequence Sequence of Rluc-H1-shRNA neo insert
tctaggtaaccgatatccctgcaggggtgacctgcacgtctagggcgcagtagtccagggtttccttgatgatgtcatacttatcctgtcccttttttttccacagctcgcggttgaggacaaactcttcgcggtctttccagtactcctgcaggtgactgactgagtcgagatctgcgatctaagtaagcttggcattccggtactgttggtaaagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctagaccggttcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcggatccgtcgacttaattaaggccagggatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattaagcttatttaaataggccggccataacttcgtataatgtatgctatacgaagttatggatcctcacagtaggtggcatcgttcctttctgactgcccgccccccgcatgccgtcccgcgatattgagctccgaacctctcgccctgccgccgccggtgctccgtcgccgccgcgccgccatggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactctttcccaggattccaattcagcgggagccacctgatgaagcttgatcgggtggctctcgctgagttggaatccatttttttctagactcgagataacttcgtataatgtatgctatacgaagttatggcgcgccggtaaccgaagttcctatactttctagagaataggaacttcggaataggaacttcttaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcccgcgccgaagttcctatactttctagagaataggaacttcggaataggaacttcaagcttaagcgc

SEQ ID NO: 6 - DNA, 15199 bp, Artificial Sequence
  Description of Artificial Sequence Targeting vector for Rosa26 locus with Rluc-H1-shRNA neo insert
ctagggataacagggtaatatagccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctaggtaaccgatatccctgcaggggtgacctgcacgtctagggcgcagtagtccagggtttccttgatgatgtcatacttatcctgtcccttttttttccacagctcgcggttgaggacaaactcttcgcggtctttccagtactcctgcaggtgactgactgagtcgagatctgcgatctaagtaagcttggcattccggtactgttggtaaagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctagaccggttcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcggatccgtcgacttaattaaggccagggatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattaagcttatttaaataggccggccataacttcgtataatgtatgctatacgaagttatggatcctcacagtaggtggcatcgttcctttctgactgcccgccccccgcatgccgtcccgcgatattgagctccgaacctctcgccctgccgccgccggtgctccgtcgccgccgcgccgccatggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactctttcccaggattccaattcagcgggagccacctgatgaagcttgatcgggtggctctcgctgagttggaatccatttttttctagactcgagataacttcgtataatgtatgctatacgaagttatggcgcgccggtaaccgaagttcctatactttctagagaataggaacttcggaataggaacttcttaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcccgcgccgaagttcctatactttctagagaataggaacttcggaataggaacttcaagcttaagcgctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagcacgtgttaactcgagctgcaggattcgagggccccggcaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatgcaccgtccttgccatcatggcctcgtaccccggccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgaggggatcgatccgtcctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctaggccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgtcgagatctagatatcgatggccatagagttacg

SEQ ID NO: 7 - DNA, 4640 bp, Artificial Sequence
  Description of Artificial Sequence Sequence of Rluc-U6-shRNA neo insert
tctaggtaaccgatatccctgcaggggtgacctgcacgtctagggcgcagtagtccagggtttccttgatgatgtcatacttatcctgtcccttttttttccacagctcgcggttgaggacaaactcttcgcggtctttccagtactcctgcaggtgactgactgagtcgagatctgcgatctaagtaagcttggcattccggtactgttggtaaagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctagaccggttcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcggatccgtcgacttaattaaggccagggatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattaagcttatttaaataggccggccataacttcgtataatgtatgctatacgaagttatggatccagtggaaagacgcgcaggcaaaacgcaccacgtgacggagcgtgaccgcgcgccgagcccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggattccaattcagcgggagccacctgatgaagcttgatcgggtggctctcgctgagttggaatccatttttttctagactcgagataacttcgtataatgtatgctatacgaagttatggcgcgccggtaaccgaagttcctatactttctagagaataggaacttcggaataggaacttcttaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcccgcgccgaagttcctatactttctagagaataggaacttcggaataggaacttcaagcttaagcgc

SEQ ID NO: 8 - DNA, 15174 bp, Artificial Sequence
  Description of Artificial Sequence Targeting vector for Rosa26 locus with a Rluc-U6-shRNA neo insert
ctagggataacagggtaatatagccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctaggtaaccgatatccctgcaggggtgacctgcacgtctagggcgcagtagtccagggtttccttgatgatgtcatacttatcctgtcccttttttttccacagctcgcggttgaggacaaactcttcgcggtctttccagtactcctgcaggtgactgactgagtcgagatctgcgatctaagtaagcttggcattccggtactgttggtaaagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctagaccggttcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcggatccgtcgacttaattaaggccagggatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattaagcttatttaaataggccggccataacttcgtataatgtatgctatacgaagttatggatccagtggaaagacgcgcaggcaaaacgcaccacgtgacggagcgtgaccgcgcgccgagcccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggattccaattcagcgggagccacctgatgaagcttgatcgggtggctctcgctgagttggaatccatttttttctagactcgagataacttcgtataatgtatgctatacgaagttatggcgcgccggtaaccgaagttcctatactttctagagaataggaacttcggaataggaacttcttaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcccgcgccgaagttcctatactttctagagaataggaacttcggaataggaacttcaagcttaagcgctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagcacgtgttaactcgagctgcaggattcgagggccccggcaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatgcaccgtccttgccatcatggcctcgtaccccggccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgaggggatcgatccgtcctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctaggccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgtcgagatctagatatcgatggccatagagttacg

SEQ ID NO: 9 - DNA, 4641 bp, Artificial Sequence
  Description of Artificial Sequence Rluc tet01 insert
tctaggtaaccgatatccctgcaggggtgacctgcacgtctagggcgcagtagtccagggtttccttgatgatgtcatacttatcctgtcccttttttttccacagctcgcggttgaggacaaactcttcgcggtctttccagtactcctgcaggtgactgactgagtcgagatctgcgatctaagtaagcttggcattccggtactgttggtaaagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctagaccggttcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcggatccgtcgacttaattaaggccagggatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattaagcttatttaaataggccggccataacttcgtataatgtatgctatacgaagttatggatccagtggaaagacgcgcaggcaaaacgcaccacgtgacggagcgtgaccgcgcgccgagcccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtactctatcattgatagagttatatatcttgtggaaaggacgaaacaccgggattccaattcagcgggagccacctgatgaagcttgatcgggtggctctcgctgagttggaatccatttttttctagactcgagataacttcgtataatgtatgctatacgaagttatggcgcgccggtaaccgaagttcctatactttctagagaataggaacttcggaataggaacttcttaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcccgcgccgaagttcctatactttctagagaataggaacttcggaataggaacttcaagcttaagcgc

SEQ ID NO: 10 - DNA, 4640 bp, Artificial Sequence
  Description of Artificial Sequence Rluc tet02 insert
tctaggtaaccgatatccctgcaggggtgacctgcacgtctagggcgcagtagtccagggtttccttgatgatgtcatacttatcctgtcccttttttttccacagctcgcggttgaggacaaactcttcgcggtctttccagtactcctgcaggtgactgactgagtcgagatctgcgatctaagtaagcttggcattccggtactgttggtaaagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctagaccggttcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcggatccgtcgacttaattaaggccagggatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattaagcttatttaaataggccggccataacttcgtataatgtatgctatacgaagttatggatccagtggaaagacgcgcaggcaaaacgcaccacgtgacggagcgtgaccgcgcgccgagcccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatctccctatcagtgatagagaaagggattccaattcagcgggagccacctgatgaagcttgatcgggtggctctcgctgagttggaatccatttttttctagactcgagataacttcgtataatgtatgctatacgaagttatggcgcgccggtaaccgaagttcctatactttctagagaataggaacttcggaataggaacttcttaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcccgcgccgaagttcctatactttctagagaataggaacttcggaataggaacttcaagcttaagcgc

SEQ ID NO: 11 - DNA, 3387 bp, Artificial Sequence
  Description of Artificial Sequence H1 tet5′ shRNA vector
agctcgatatcggccggccataacttcgtataatgtatgctatacgaagttatggatcctcacagtaggtggcatcgttcctttctgactgcccgccccccgcatgccgtcccgcgatattgagctccgaacctctcgccctgccgccgccggtgctccgtcgccgccgcgccgccatggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaattccctatcagtgatagagattataagttctgtatgagaccactctttcccaggattccaattcagcgggagccacctgatgaagcttgatcgggtggctctcgctgagttggaatccatttttttctaggataacttcgtataatgtatgctatacgaagttatggcgcgccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgg

SEQ ID NO: 12 - DNA, 3387 bp, Artificial Sequence
  Description of Artificial Sequence H1 tet3′ shRNA vector
agctcgatatcggccggccataacttcgtataatgtatgctatacgaagttatggatcctcacagtaggtggcatcgttcctttctgactgcccgccccccgcatgccgtcccgcgatattgagctccgaacctctcgccctgccgccgccggtgctccgtcgccgccgcgccgccatggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagtccctatcagtgatagagattcccaggattccaattcagcgggagccacctgatgaagcttgatcgggtggctctcgctgagttggaatccatttttttctaggataacttcgtataatgtatgctatacgaagttatggcgcgccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgg

SEQ ID NO: 13 - DNA, 3387 bp, Artificial Sequence
  Description of Artificial Sequence H1 tet5′ + 3′ shRNA vector
agctcgatatcggccggccataacttcgtataatgtatgctatacgaagttatggatcctcacagtaggtggcatcgttcctttctgactgcccgccccccgcatgccgtcccgcgatattgagctccgaacctctcgccctgccgccgccggtgctccgtcgccgccgcgccgccatggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaattccctatcagtgatagagattataagtccctatcagtgatagagattcccaggattccaattcagcgggagccacctgatgaagcttgatcgggtggctctcgctgagttggaatccatttttttctaggataacttcgtataatgtatgctatacgaagttatggcgcgccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgg

SEQ ID NO: 14 - DNA, 7209 bp, Artificial Sequence
  Description of Artificial Sequence Fluc-GAGGS-tetR-hygro insert
tctaggtaaccgatatccctgcaggggtgacctgcacgtctagggcgcagtagtccagggtttccttgatgatgtcatacttatcctgtcccttttttttccacagctcgcggttgaggacaaactcttcgcggtctttccagtactcctgcaggtgactgactgagtcgagatctgcgatctaagtaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagaccggttcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgcccccgatccgtcgacgcccgggctagcttgcatgcctgcaggttttcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggcctccggagcgatcgccgatccgcctagcattcaaccatggctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcgtaaccggttcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgcccccgggtaccgagctcgaattgttaattaaggccatagcggccgccctgaggccgcgggcgatcgcctaggggtaaccgaagttcctatactttctagagaataggaacttcggaataggaacttcttataatctagaagatctggatccacgattcgagggcccctgcaggtcaattctaccgggtaggggaggcgcttttcccaaggcagtctggagcatgcgctttagcagccccgctggcacttggcgctacacaagtggcctctggcctcgcacacattccacatccaccggtagcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcgaaggtcctcccgaggcccggcattctcgcacgcttcaaaagcgcacgtctgccgcgctgttctcctcttcctcatctccgggcctttcgacgatccagccgccaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgccatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtcgatgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcctgcaggaattcatatgaagttcctatactttctagagaataggaacttcggaataggaacttcaaaatgtcgcggcgcgccggtaaccgaagttcctatactttctagagaataggaacttcggaataggaacttcaagcttaagcgc

SEQ ID NO: 15 - DNA, 7014 bp, Artificial Sequence
  Description of Artificial Sequence GAGGS-Fluc
tagcccgggctagcttgcatgcctgcaggttttcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggcctccggagcgatcgccggtccgcctaggcaattgtttaaactcgaggatctgcgatctaagtaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatctgaacgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgataggtaccgagctcttacgcgtgc

SEQ ID NO: 16 - DNA, 5430 bp, Artificial Sequence
  Description of Artificial Sequence GAGGS-tetR
ccgggctagcttgcatgcctgcaggttttcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggcctccggagcgatcgccgatccgcctagcattcaaccatggctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcgtaaccggttcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgcccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctagc

SEQ ID NO: 17 - DNA, 7332 bp, Artificial Sequence
  Description of Artificial Sequence CMV-LacZ
aaacagtccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctgactctagacttaattaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggccgcaattcccggggatcgaaagagcctgctaaagcaaaaaagaagtcaccatgtcgtttactttgaccaacaagaacgtgattttcgttgccggtctgggaggcattggtctggacaccagcaaggagctgctcaagcgcgatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattacagctgagcgccggtcgctaccattaccagttggtctggtgtcaaaaataataataaccgggcaggccatgtctgcccgtatttcgcgtaaggaaatccattatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcggcctcgactctaggcggccgcgtcgacctcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgcccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctagcccgggctagcttgcatgcctgcaggttt

SEQ ID NO: 18 - DNA, 5878 bp, Artificial Sequence
  Description of Artificial Sequence CAGGS-cre
ttcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagtaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggcctcgaccatgcccaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagccattaacgcgtaaatgattgcagatccactagttctagggccgcgtcgacctcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgcccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctagcccgggctagcttgcatgcctgcaggtt

SEQ ID NO: 19 - DNA, 47 bp, Artificial Sequence
  Description of Artificial Sequence DNA encoding an shRNA directed against the CDH-1 gene
tgagaagtctcccagtcagttcaagagactgactgggagacttctca

SEQ ID NO: 20 - DNA, 47 bp, Artificial Sequence
  Description of Artificial Sequence DNA encoding an shRNA directed against the p53 gene
gactccagtggtaatctacttcaagagagtagattaccactggagtc

SEQ ID NO: 21 - DNA, 47 bp, Artificial Sequence
  Description of Artificial Sequence DNA encoding an shRNA directed against the CDC20 gene
cggcaggactccgggccgattcaagagatcggcccggagtcctgccg

SEQ ID NO: 22 - DNA, 47 bp, Artificial Sequence
  Description of Artificial Sequence DNA encoding an shRNA directed against the CYLD gene
cctcatgcagttctctttgttcaagagacaaagagaactgcatgagg

SEQ ID NO: 23 - DNA, 50 bp, Artificial Sequence
  Description of Artificial Sequence DNA encoding an shRNA directed against the RAS-GAP gene
aagatgaagccactccctatttcaagagaaaatagggagtggcttcatct

SEQ ID NO: 24 - DNA, 41 bp, Artificial Sequence
  Description of Artificial Sequence DNA encoding an shRNA directed against the tubulin gene
gacagagccaagtggactcacagagtccacttggctctgtc

SEQ ID NO: 25 - DNA, 42 bp, Artificial Sequence
  Description of Artificial Sequence DNA encoding an shRNA directed against the lamin gene
ctggacttccagaagaacattcgtgttcttctggaagtccag

SEQ ID NO: 26 - DNA, 47 bp, Artificial Sequence
  Description of Artificial Sequence DNA encoding an shRNA directed against the gene for the ubiquitin carboxyl-terminal hydrolase 12
gagattggtccagaacagtttcaagagaactgttctggaccaatctc

SEQ ID NO: 27 - DNA, 47 bp, Artificial Sequence
  Description of Artificial Sequence DNA encoding an shRNA directed against the gene for the ubiquitin carboxyl-terminal hydrolase 12
gcccttccgatcatggtagttcaagagactaccatgatcggaagggc

SEQ ID NO: 28 - DNA, 47 bp, Artificial Sequence
  Description of Artificial Sequence DNA encoding an shRNA directed against the gene for the ubiquitin carboxyl-terminal hydrolase 12
tctttagaattcttaagtattcaagagatacttaagaattctaaaga

SEQ ID NO: 29 - DNA, 47 bp, Artificial Sequence
  Description of Artificial Sequence DNA encoding an shRNA directed against the gene of the ubiquitin carboxyl-terminal hydrolase
cattagctatatcaacatgttcaagagacatgttgatatagctaatg

SEQ ID NO: 30 - DNA, 47 bp, Artificial Sequence
  Description of Artificial Sequence DNA encoding an shRNA directed against the gene for the ubiquitin carboxyl-terminal hydrolase11
accacaaacggcggaacgattcaagagatcgttccgccgtttgtggt

SEQ ID NO: 31 - DNA, 47 bp, Artificial Sequence
  Description of Artificial Sequence DNA encoding an shRNA directed against the gene for the ubiquitin carboxyl-terminal hydrolase 11
gagggtcttggaggtcttcttcaagagagaagacctccaagaccctc

SEQ ID NO: 32 - DNA, 47 bp, Artificial Sequence
  Description of Artificial Sequence DNA encoding an shRNA directed against the gene for the ubiquitin carboxyl-terminal hydrolase 11
gtccatgcccagccgtacattcaagagatgtacggctgggcatggac

SEQ ID NO: 33 - DNA, 47 bp, Artificial Sequence
  Description of Artificial Sequence DNA encoding an shRNA directed against the gene for the ubiquitin carboxyl-terminal hydrolase 11
gctggacaccctcgtggagttcaagagactccacgagggtgtccagc

SEQ ID NO: 34 - DNA, 47 bp, Artificial Sequence
  Description of Artificial Sequence DNA encoding an shRNA directed against the gene for the ubiquitin carboxyl-terminal hydrolase 10
gaatatcagagaattgagtttcaagagaactcaattctctgatattc

SEQ ID NO: 35 - DNA, 47 bp, Artificial Sequence
  Description of Artificial Sequence DNA encoding an shRNA directed against the gene for the ubiquitin carboxyl-terminal hydrolase 10
tggacttcatgaggaaatgttcaagagacatttcctcatgaagtcca

SEQ ID NO: 36 - DNA, 47 bp, Artificial Sequence
  Description of Artificial Sequence DNA encoding an shRNA directed against the  gene for the ubiquitin carboxyl-terminal hydrolase 10
tattgaatatcctgtggacttcaagagagtccacaggatattcaata

SEQ ID NO: 37 - DNA, 47 bp, Artificial Sequence
  Description of Artificial Sequence DNA encoding an shRNA directed against the gene for the ubiquitin carboxyl-terminal hydrolase 10
ttgtactgagagaaactgcttcaagagagcagtttctctcagtacaa

SEQ ID NO: 38 - DNA, 47 bp, Artificial Sequence
  Description of Artificial Sequence DNA encoding an shRNA directed against the HAUSP gene
gatcaatgataggtttgaattcaagagattcaaacctatcattgatc

SEQ ID NO: 39 - DNA, 47 bp, Artificial Sequence
  Description of Artificial Sequence DNA encoding an shRNA directed against the HAUSP gene
ggagtttgagaagtttaaattcaagagatttaaacttctcaaactcc

SEQ ID NO: 40 - DNA, 47 bp, Artificial Sequence
  Description of Artificial Sequence DNA encoding an shRNA directed against the HAUSP gene
gaactcctcgcttgctgagttcaagagactcagcaagcgaggagttc

SEQ ID NO: 41 - DNA, 47 bp, Artificial Sequence
  Description of Artificial Sequence DNA encoding an shRNA directed against the HAUSP gene
ccgaatttaacagagagaattcaagagattctctctgttaaattcgg

SEQ ID NO: 42 - DNA, 47 bp, Artificial Sequence
  Description of Artificial Sequence DNA encoding an shRNA directed against the gene for the ubiquitin carboxyl-terminal hydrolase 8
gacagcagaagaatgcagattcaagagatctgcattcttctgctgtc

SEQ ID NO: 43 - DNA, 47 bp, Artificial Sequence
  Description of Artificial Sequence DNA encoding an shRNA directed against the gene for the ubiquitin carboxyl-terminal hydrolase 8
ataaagctcaacgagaaccttcaagagaggttctcgttgagctttat

SEQ ID NO: 44 - DNA, 47 bp, Artificial Sequence
  Description of Artificial Sequence DNA encoding an shRNA directed against the gene for the ubiquitin carboxyl-terminal hydrolase 8
ggtgaagtggcagaagaatttcaagagaattcttctgccacttcacc

SEQ ID NO: 45 - DNA, 47 bp, Artificial Sequence
  Description of Artificial Sequence DNA encoding an shRNA directed against the gene for the ubiquitin carboxyl-terminal hydrolase 8
gtattgcagtaatcatcacttcaagagagtgatgattactgcaatac

SEQ ID NO: 46 - DNA, 47 bp, Artificial Sequence
  Description of Artificial Sequence DNA encoding an shRNA directed against the FLJ10785 gene
gatatggggttccatgtcattcaagagatgacatggaaccccatatc

SEQ ID NO: 47 - DNA, 47 bp, Artificial Sequence
  Description of Artificial Sequence DNA encoding an shRNA directed against the FLJ10785 gene
ggagacatggttcttagtgttcaagagacactaagaaccatgtctcc

SEQ ID NO: 48 - DNA, 47 bp, Artificial Sequence
  Description of Artificial Sequence DNA encoding an shRNA directed against the FLJ10785 gene
agcaccaagttcgtctcagttcaagagactgagacgaacttggtgct

SEQ ID NO: 49 - DNA, 47 bp, Artificial Sequence
  Description of Artificial Sequence DNA encoding an shRNA directed against the FLJ10785 gene
gatgcaacactgaaagaacttcaagagagttctttcagtgttgcatc

SEQ ID NO: 50 - DNA, 47 bp, Artificial Sequence
  Description of Artificial Sequence DNA encoding an shRNA directed against the KIAA0710 gene
gtcaatggcagtgatgatattcaagagatatcatcactgccattgac

SEQ ID NO: 51 - DNA, 47 bp, Artificial Sequence
  Description of Artificial Sequence DNA encoding an shRNA directed against the KIAA0710 gene
cctgctagctgcctgtggcttcaagagagccacaggcagctagcagg

SEQ ID NO: 52 - DNA, 47 bp, Artificial Sequence
  Description of Artificial Sequence DNA encoding an shRNA directed against the KIAA0710 gene
ccacctttgccagaaggagttcaagagactccttctggcaaaggtgg

SEQ ID NO: 53 - DNA, 47 bp, Artificial Sequence
  Description of Artificial Sequence DNA encoding an shRNA directed against the KIAA0710 gene
ccctattgaggcaagtgtcttcaagagagacacttgcctcaataggg

SEQ ID NO: 54 - DNA, 47 bp, Artificial Sequence
  Description of Artificial Sequence DNA encoding an shRNA directed against the genes FLJ12552/FLJ14256
gaaggaaaacttgctgacgttcaagagacgtcagcaagttttccttc

SEQ ID NO: 55 - DNA, 47 bp, Artificial Sequence
  Description of Artificial Sequence DNA encoding an shRNA directed against the FLJ12552/FLJ14256 genes
ctcacctgggtccatgagattcaagagatctcatggacccaggtgag

SEQ ID NO: 56 - DNA, 47 bp, Artificial Sequence
  Description of Artificial Sequence DNA encoding an shRNA directed against the FLJ12552/FLJ14256 genes
gctgtcttaccgtgtggtcttcaagagagaccacacggtaagacagc

SEQ ID NO: 57 - DNA, 47 bp, Artificial Sequence
  Description of Artificial Sequence DNA encoding an shRNA directed against the FLJ12552/FLJ14256 genes
cctggaccgcatgtatgacttcaagagagtcatacatgcggtccagg

SEQ ID NO: 58 - DNA, 47 bp, Artificial Sequence; identical to SEQ ID NO: 50
  Description of Artificial Sequence DNA encoding an shRNA directed against the KIAA1203 gene

SEQ ID NO: 59 - DNA, 47 bp, Artificial Sequence; identical to SEQ ID NO: 51
  Description of Artificial Sequence DNA encoding an shRNA directed against the KIAA1203 gene

SEQ ID NO: 60 - DNA, 47 bp, Artificial Sequence; identical to SEQ ID NO: 52
  Description of Artificial Sequence DNA encoding an shRNA directed against the KIAA1203 gene

SEQ ID NO: 61 - DNA, 47 bp, Artificial Sequence; identical to SEQ ID NO: 53
  Description of Artificial Sequence DNA encoding an shRNA directed against the KIAA1203 gene

SEQ ID NO: 62 - DNA, 47 bp, Artificial Sequence
  Description of Artificial Sequence DNA encoding an shRNA directed against the FLJ23277 gene
ggaaatccgaattgcttggttcaagagaccaagcaattcggatttcc

SEQ ID NO: 63 - DNA, 47 bp, Artificial Sequence
  Description of Artificial Sequence DNA encoding an shRNA directed against the FLJ23277 gene
cacatttcttcaagtgtggttcaagagaccacacttgaagaaatgtg

SEQ ID NO: 64 - DNA, 47 bp, Artificial Sequence
  Description of Artificial Sequence DNA encoding an shRNA directed against the FLJ23277 gene
cagcaggatgctcaagaatttcaagagaattcttgagcatcctgctg

SEQ ID NO: 65 - DNA, 47 bp, Artificial Sequence
  Description of Artificial Sequence DNA encoding an shRNA directed against the FLJ23277 gene
gctgaatacctacattggcttcaagagagccaatgtaggtattcagc

SEQ ID NO: 66 - DNA, 47 bp, Artificial Sequence
  Description of Artificial Sequence DNA encoding an shRNA directed against the FLJ14914 (similar to UBP4) gene
gggcttgtgcctggccttgttcaagagacaaggccaggcacaagccc

SEQ ID NO: 67 - DNA, 47 bp, Artificial Sequence
  Description of Artificial Sequence DNA encoding an shRNA directed against the FLJ14914 (similar to UBP4) gene
gccttgtcctgccaagaagttcaagagacttcttggcaggacaaggc

SEQ ID NO: 68 - DNA, 47 bp, Artificial Sequence
  Description of Artificial Sequence DNA encoding an shRNA directed against the FLJ14914 (similar to UBP4) gene
gattgaagccaagggaacgttcaagagacgttcccttggcttcaatc

SEQ ID NO: 69 - DNA, 47 bp, Artificial Sequence
  Description of Artificial Sequence DNA encoding an shRNA directed against the FLJ14914 (similar to UBP4) gene
tggcgcctgctccccatctttcaagagaagatggggagcaggcgcca

SEQ ID NO: 70 - DNA, 47 bp, Artificial Sequence
  Description of Artificial Sequence DNA encoding an shRNA directed against the gene for the ubiquitin carboxyl-terminal hydrolase isozyme L5
gaaccagcaggctctgtggttcaagagaccacagagcctgctggttc

SEQ ID NO: 71 - DNA, 47 bp, Artificial Sequence
  Description of Artificial Sequence DNA encoding an shRNA directed against the gene for the ubiquitin carboxyl-terminal hydrolase isozyme L5
ggaagcataattatctgccttcaagagaggcagataattatgcttcc

SEQ ID NO: 72 - DNA, 47 bp, Artificial Sequence
  Description of Artificial Sequence DNA encoding an shRNA directed against the gene for the ubiquitin carboxyl-terminal hydrolase isozyme L5
agaagaagatgcttttcacttcaagagagtgaaaagcatcttcttct

SEQ ID NO: 73 - DNA, 47 bp, Artificial Sequence
  Description of Artificial Sequence DNA encoding an shRNA directed against the gene for the ubiquitin carboxyl-terminal hydrolase isozyme L5
cttgcagaggaggaacccattcaagagatgggttcctcctctgcaag

SEQ ID NO: 74 - DNA, 47 bp, Artificial Sequence
  Description of Artificial Sequence DNA encoding an shRNA directed against the gene for the ubiquitin carboxyl-terminal hydrolase isozyme L3
gcaaacaatcagcaatgccttcaagagaggcattgctgattgtttgc

SEQ ID NO: 75 - DNA, 47 bp, Artificial Sequence
  Description of Artificial Sequence DNA encoding an shRNA directed against the gene for the ubiquitin carboxyl-terminal hydrolase isozyme L3
ttggactgattcatgctatttcaagagaatagcatgaatcagtccaa

SEQ ID NO: 76 - DNA, 47 bp, Artificial Sequence
  Description of Artificial Sequence DNA encoding an shRNA directed against the gene for the ubiquitin carboxyl-terminal hydrolase isozyme L3
ctggcaattcgttgatgtattcaagagatacatcaacgaattgccag

SEQ ID NO: 77 - DNA, 47 bp, Artificial Sequence
  Description of Artificial Sequence DNA encoding an shRNA directed against the gene for the ubiquitin carboxyl-terminal hydrolase isozyme L3
ttagatgggcggaagccatttcaagagaatggcttccgcccatctaa

SEQ ID NO: 78 - DNA, 47 bp, Artificial Sequence
  Description of Artificial Sequence DNA encoding an shRNA directed against the gene for the ubiquitin carboxyl-terminal hydrolase isozyme L1
gaggagtctctgggctcggttcaagagaccgagcccagagactcctc

SEQ ID NO: 79 - DNA, 47 bp, Artificial Sequence
  Description of Artificial Sequence DNA encoding an shRNA directed against the gene for the ubiquitin carboxyl-terminal hydrolase isozyme L1
gagctgaagggacaagaagttcaagagacttcttgtcccttcagctc

SEQ ID NO: 80 - DNA, 47 bp, Artificial Sequence
  Description of Artificial Sequence DNA encoding an shRNA directed against the gene for the ubiquitin carboxyl-terminal hydrolase isozyme L1
tgtcgggtagatgacaaggttcaagagaccttgtcatctacccgaca

SEQ ID NO: 81 - DNA, 47 bp, Artificial Sequence
  Description of Artificial Sequence DNA encoding an shRNA directed against the gene for the ubiquitin carboxyl-terminal hydrolase isozyme L1
cacagctgttcttctgttcttcaagagagaacagaagaacagctgtg

SEQ ID NO: 82 - DNA, 47 bp, Artificial Sequence
  Description of Artificial Sequence DNA encoding an shRNA directed against the KIAA1891/FLJ25263 genes
gtggaagcctttacagatcttcaagagagatctgtaaaggcttccac

SEQ ID NO: 83 - DNA, 47 bp, Artificial Sequence
  Description of Artificial Sequence DNA encoding an shRNA directed against the KIAA1891/FLJ25236 genes
caacagctgccttcatctgttcaagagacagatgaaggcagctgttg

SEQ ID NO: 84 - DNA, 47 bp, Artificial Sequence
  Description of Artificial Sequence DNA encoding an shRNA directed against the KIAA1891/FLJ25263 genes
ccataggcagtcctcctaattcaagagattaggaggactgcctatgg

SEQ ID NO: 85 - DNA, 47 bp, Artificial Sequence
  Description of Artificial Sequence DNA encoding an shRNA directed against the KIAA1891/FLJ25263 genes
tgtatcactgccactggttttcaagagaaaccagtggcagtgataca

SEQ ID NO: 86 - DNA, 47 bp, Artificial Sequence
  Description of Artificial Sequence DNA encoding an shRNA directed against the FLJ14528 (similar to UBP8) gene
catgttgggcagctgcagcttcaagagagctgcagctgcccaacatg

SEQ ID NO: 87 - DNA, 47 bp, Artificial Sequence
  Description of Artificial Sequence DNA encoding an shRNA directed against the FLJ14528 (similar to UBP8) gene
cacaactggagacctgaagttcaagagacttcaggtctccagttgtg

SEQ ID NO: 88 - DNA, 47 bp, Artificial Sequence
  Description of Artificial Sequence DNA encoding an shRNA directed against the FLJ14528 (similar to UBP8) gene
gtatgcctccaagaaagagttcaagagactctttcttggaggcatac

SEQ ID NO: 89 - DNA, 47 bp, Artificial Sequence
  Description of Artificial Sequence DNA encoding an shRNA directed against the FLJ14528 (similar to UBP8) gene
cttcacagtacatttctctttcaagagaagagaaatgtactgtgaag

SEQ ID NO: 90 - DNA, 47 bp, Artificial Sequence
  Description of Artificial Sequence DNA encoding an shRNA directed against the gene for the U4/U6 TRI snRNP 65 kDa protein
gtactttcaaggccggggtttcaagagaaccccggccttgaaagtac

SEQ ID NO: 91 - DNA, 47 bp, Artificial Sequence
  Description of Artificial Sequence DNA encoding an shRNA directed against the gene for the U4/U6 TRI snRNP 65 kDa protein
cttggacaagcaagccaaattcaagagatttggcttgcttgtccaag

SEQ ID NO: 92 - DNA, 47 bp, Artificial Sequence
  Description of Artificial Sequence DNA encoding an shRNA directed against the gene for the U4/U6 TRI snRNP 65 kDa protein
gactattgtgactgatgttttcaagagaaacatcagtcacaatagtc

SEQ ID NO: 93 - DNA, 47 bp, Artificial Sequence
  Description of Artificial Sequence DNA encoding an shRNA directed against the gene for the U4/U6 TRI snRNP 65kDa protein
ggagaactttctgaagcgcttcaagagagcgcttcagaaagttctcc

SEQ ID NO: 94 - DNA, 47 bp, Artificial Sequence
  Description of Artificial Sequence DNA encoding an shRNA for the XM_089437 gene
gacgagagaaaccttcaccttcaagagaggtgaaggtttctctcgtc

SEQ ID NO: 95 - DNA, 47 bp, Artificial Sequence
  Description of Artificial Sequence DNA encoding an shRNA directed against the XM_089437 gene
acattattctacattctttttcaagagaaaagaatgtagaataatgt

SEQ ID NO: 96 - DNA, 47 bp, Artificial Sequence
  Description of Artificial Sequence DNA encoding an shRNA directed against the XM_089437 gene
agattcgcaaatggatgtattcaagagatacatccatttgcgaatct

SEQ ID NO: 97 - DNA, 47 bp, Artificial Sequence
  Description of Artificial Sequence DNA encoding an shRNA directed against the XM_089437 gene
cattcccaccatgagtctgttcaagagacagactcatggtgggaatg

SEQ ID NO: 98 - DNA, 47 bp, Artificial Sequence
  Description of Artificial Sequence DNA encoding an shRNA directed against the KIAA1453 gene
gatcgcccgacacttccgcttcaagagagcggaagtgtcgggcgatc

SEQ ID NO: 99 - DNA, 47 bp, Artificial Sequence
  Description of Artificial Sequence DNA encoding an shRNA directed against the KIAA1453 gene
ccagcaggcctacgtgctgttcaagagacagcacgtaggcctgctgg

SEQ ID NO: 100 - DNA, 47 bp, Artificial Sequence
  Description of Artificial Sequence DNA encoding an shRNA directed against the KIAA1453 gene
gccagctcctccacagcacttcaagagagtgctgtggaggagctggc

SEQ ID NO: 101 - DNA, 47 bp, Artificial Sequence
  Description of Artificial Sequence DNA encoding an shRNA directed against the KIAA1453 gene
cgccgccaagtggagcagattcaagagatctgctccacttggcggcg

SEQ ID NO: 102 - DNA, 47 bp, Artificial Sequence
  Description of Artificial Sequence DNA encoding an shRNA directed against the FLJ12697 gene
gaagatgcccatgaattccttcaagagaggaattcatgggcatcttc

SEQ ID NO: 103 - DNA, 47 bp, Artificial Sequence
  Description of Artificial Sequence DNA encoding an shRNA directed against the FLJ12697 gene
caaacaggctgcgccaggcttcaagagagcctggcgcagcctgtttg

SEQ ID NO: 104 - DNA, 47 bp, Artificial Sequence
  Description of Artificial Sequence DNA encoding an shRNA directed against the FLJ12697 gene
acggcctagcgcctgatggttcaagagaccatcaggcgctaggccgt

SEQ ID NO: 105 - DNA, 47 bp, Artificial Sequence
  Description of Artificial Sequence DNA encoding an shRNA directed against the FLJ12697 gene
ctgtaacctctctgatcggttcaagagaccgatcagagaggttacag

SEQ ID NO: 106 - DNA, 47 bp, Artificial Sequence
  Description of Artificial Sequence DNA encoding an shRNA directed against the gene for the ubiquitin specific protease (USP18)
tctgtcagtccatcctggcttcaagagagccaggatggactgacaga

SEQ ID NO: 107 - DNA, 47 bp, Artificial Sequence
  Description of Artificial Sequence DNA encoding an shRNA directed against the gene for the ubiquitin specific protease (USP18)
tgaagcgagagtcttgtgattcaagagatcacaagactctcgcttca

SEQ ID NO: 108 - DNA, 47 bp, Artificial Sequence
  Description of Artificial Sequence DNA encoding an shRNA directed against the gene for the ubiquitin specific protease (USP18)
gatggagtgctaatggaaattcaagagatttccattagcactccatc

SEQ ID NO: 109 - DNA, 47 bp, Artificial Sequence
  Description of Artificial Sequence DNA encoding an shRNA directed against the gene for the ubiquitin specific protease (USP18)
ccttcagagattgacacgcttcaagagagcgtgtcaatctctgaagg

SEQ ID NO: 110 - DNA, 47 bp, Artificial Sequence
  Description of Artificial Sequence DNA encoding an shRNA directed against the gene for the ubiquitin carboxyl-terminal hydrolase 20
cctgaccacgttccgactgttcaagagacagtcggaacgtggtcagg

SEQ ID NO: 111 - DNA, 47 bp, Artificial Sequence
  Description of Artificial Sequence DNA encoding an shRNA directed against the gene for the ubiquitin carboxyl-terminal hydrolase 20
gagttccttcgctgcctgattcaagagatcaggcagcgaaggaactc

SEQ ID NO: 112 - DNA, 47 bp, Artificial Sequence
  Description of Artificial Sequence DNA encoding an shRNA directed against the gene for the ubiquitin carboxyl-terminal hydrolase 20
gactgccttgctgccttctttcaagagaagaaggcagcaaggcagtc

SEQ ID NO: 113 - DNA, 47 bp, Artificial Sequence
  Description of Artificial Sequence DNA encoding an shRNA directed against the gene for the ubiquitin carboxyl-terminal hydrolase 20
cgccgagggctacgtactcttcaagagagagtacgtagccctcggcg

SEQ ID NO: 114 - DNA, 47 bp, Artificial Sequence
  Description of Artificial Sequence DNA encoding an shRNA directed against the gene for the ubiquitin carboxyl-terminal hydrolase 24
ggcgagaagaaaggactgtttcaagagaacagtcctttcttctcgcc

SEQ ID NO: 115 - DNA, 47 bp, Artificial Sequence
  Description of Artificial Sequence DNA encoding an shRNA directed against the gene for the ubiquitin carboxyl terminal hydrolase 24
ggacgagaattgataaagattcaagagatctttatcaattctcgtcc

SEQ ID NO: 116 - DNA, 47 bp, Artificial Sequence
  Description of Artificial Sequence DNA encoding an shRNA directed against the gene for the ubiquitin carboxyl-terminal hydrolase 24
gcacgagaatttgggaatcttcaagagagattcccaaattctcgtgc

SEQ ID NO: 117 - DNA, 47 bp, Artificial Sequence
  Description of Artificial Sequence DNA encoding an shRNA directed against the gene for the ubiquitin carboxyl-terminal hydrolase 24
ctacttcatgaaatattggttcaagagaccaatatttcatgaagtag

SEQ ID NO: 118 - DNA, 47 bp, Artificial Sequence
  Description of Artificial Sequence DNA encoding an shRNA directed against the KIAA1594 gene
gataacagcttcttgtctattcaagagatagacaagaagctgttatc

SEQ ID NO: 119 - DNA, 47 bp, Artificial Sequence
  Description of Artificial Sequence DNA encoding an shRNA directed against the KIAA1594 gene
gagaataggacatcagggcttcaagagagccctgatgtcctattctc

SEQ ID NO: 120 - DNA, 47 bp, Artificial Sequence
  Description of Artificial Sequence DNA encoding an shRNA directed against the KIAA1594 gene
cttggaagactgaacctgtttcaagagaacaggttcagtcttccaag

SEQ ID NO: 121 - DNA, 47 bp, Artificial Sequence
  Description of Artificial Sequence DNA encoding an shRNA directed against the KIAA1594 gene
caactcctttgtggatgcattcaagagatgcatccacaaaggagttg

SEQ ID NO: 122 - DNA, 47 bp, Artificial Sequence
  Description of Artificial Sequence DNA encoding an shRNA directed against the KIAA1350 gene
gatgttgtctccaaatgcattcaagagatgcatttggagacaacatc

SEQ ID NO: 123 - DNA, 47 bp, Artificial Sequence
  Description of Artificial Sequence DNA encoding an shRNA directed against the KIAA1350 gene
cgtggggactgtacctcccttcaagagagggaggtacagtccccacg

SEQ ID NO: 124 - DNA, 47 bp, Artificial Sequence
  Description of Artificial Sequence DNA encoding an shRNA direcetd against the KIAA1350 gene
gtacagcttcagaaccaagttcaagagacttggttctgaagctgtac

SEQ ID NO: 125 - DNA, 47 bp, Artificial Sequence
  Description of Artificial Sequence DNA encoding an shRNA directed against the gene for the ubiquitin carboxyl-terminal hydrolase 25
gatgatcttcagagagcaattcaagagattgctctctgaagatcatc

SEQ ID NO: 126 - DNA, 47 bp, Artificial Sequence
  Description of Artificial Sequence DNA encoding an shRNA directed against the gene for the ubiquitin carboxyl-terminal hydrolase 25
ggaacatcggaatttgcctttcaagagaaggcaaattccgatgttcc

SEQ ID NO: 127 - DNA, 47 bp, Artificial Sequence
  Description of Artificial Sequence DNA encoding an shRNA directed against the gene for the ubiquitin carboxyl-terminal hydrolase 25
gagctagtgagggactcttttcaagagaaagagtccctcactagctc

SEQ ID NO: 128 - DNA, 47 bp, Artificial Sequence
  Description of Artificial Sequence DNA encoding an shRNA directed against the gene for the ubiquitin carboxyl-terminal hydrolase 25
gcagggttctttaaggcaattcaagagattgccttaaagaaccctgc

SEQ ID NO: 129 - DNA, 47 bp, Artificial Sequence
  Description of Artificial Sequence DNA encoding an shRNA directed against the gene for the ubiquitin carboxyl-terminal hydrolase 16
tcgatgattcctctgaaacttcaagagagtttcagaggaatcatcga

SEQ ID NO: 130 - DNA, 47 bp, Artificial Sequence
  Description of Artificial Sequence DNA encoding an shRNA directed against the gene for the ubiquitin carboxyl-terminal hydrolase 16
gataatggaaatattgaacttcaagagagttcaatatttccattatc

SEQ ID NO: 131 - DNA, 47 bp, Artificial Sequence
  Description of Artificial Sequence DNA encoding an shRNA directed against the gene for the ubiquitin carboxyl-terminal hydrolase 16
gttcttcatttaaatgatattcaagagatatcatttaaatgaagaac

SEQ ID NO: 132 - DNA, 47 bp, Artificial Sequence
  Description of Artificial Sequence DNA encoding an shRNA directed against the gene for the ubiquitin carboxyl-terminal hydrolase 16
gttaacaaacacataaagtttcaagagaactttatgtgtttgttaac

SEQ ID NO: 133 - DNA, 47 bp, Artificial Sequence
  Description of Artificial Sequence DNA encoding an shRNA directed against the USP9X gene
gttagagaagattcttcgtttcaagagaacgaagaatcttctctaac

SEQ ID NO: 134 - DNA, 47 bp, Artificial Sequence
  Description of Artificial Sequence DNA encoding an shRNA directed against the USP9X gene
gttgattggacaattaaacttcaagagagtttaattgtccaatcaac

SEQ ID NO: 135 - DNA, 47 bp, Artificial Sequence
  Description of Artificial Sequence DNA encoding an shRNA directed against the USP9X gene
ggttgataccgtaaagcgcttcaagagagcgctttacggtatcaacc

SEQ ID NO: 136 - DNA, 47 bp, Artificial Sequence
  Description of Artificial Sequence DNA encoding an shRNA directed against the USP9X gene
gcaatgaaacgtccaatggttcaagagaccattggacgtttcattgc

SEQ ID NO: 137 - DNA, 47 bp, Artificial Sequence
  Description of Artificial Sequence DNA encoding an shRNA directed against the USP9Y gene
agctagagaaaattcttcgttcaagagacgaagaattttctctagct

SEQ ID NO: 138 - DNA, 47 bp, Artificial Sequence
  Description of Artificial Sequence DNA encoding an shRNA directed against the USP9Y gene
gatcctatatgatggatgattcaagagatcatccatcatataggatc

SEQ ID NO: 139 - DNA, 47 bp, Artificial Sequence
  Description of Artificial Sequence DNA encoding an shRNA directed against the USP9Y gene
gttcttcttgtcagtgaaattcaagagatttcactgacaagaagaac

SEQ ID NO: 140 - DNA, 47 bp, Artificial Sequence
  Description of Artificial Sequence DNA encoding an shRNA directed against the USP9Y gene
cttgagcttgagtgaccacttcaagagagtggtcactcaagctcaag

SEQ ID NO: 141 - DNA, 47 bp, Artificial Sequence
  Description of Artificial Sequence DNA encoding an shRNA direcetd against the gene for the ubiquitin carboxyl-terminal hydrolase 5
gaccggccagcgagtctacttcaagagagtagactcgctggccggtc

SEQ ID NO: 142 - DNA, 47 bp, Artificial Sequence
  Description of Artificial Sequence DNA encoding an shRNA directed against the gene for the ubiquitin carboxyl-terminal hydrolase 5
ggacctgggctacatctacttcaagagagtagatgtagcccaggtcc

SEQ ID NO: 143 - DNA, 47 bp, Artificial Sequence
  Description of Artificial Sequence DNA encoding an shRNA directed against the gene for the ubiquitin carboxyl-terminal hydrolase 5
ctctgtggtccaggtgctcttcaagagagagcacctggaccacagag

SEQ ID NO: 144 - DNA, 47 bp, Artificial Sequence
  Description of Artificial Sequence DNA encoding an shRNA directed against the gene for the ubiquitin carboxyl-terminal hydrolase 5
gaccacacgatttgcctcattcaagagatgaggcaaatcgtgtggtc

SEQ ID NO: 145 - DNA, 47 bp, Artificial Sequence
  Description of Artificial Sequence DNA encoding an shRNA directed against the gene for the ubiquitin carboxyl-terminal hydrolase 26
tggcttgtttattgaaggattcaagagatccttcaataaacaagcca

SEQ ID NO: 146 - DNA, 47 bp, Artificial Sequence
  Description of Artificial Sequence DNA encoding an shRNA directed against the gene for the ubiquitin carboxyl-terminal hydrolase 26
gtgaatttggggaagataattcaagagattatcttccccaaattcac

SEQ ID NO: 147 - DNA, 47 bp, Artificial Sequence
  Description of Artificial Sequence DNA encoding an shRNA directed against the gene for the ubiquitin carboxyl- terminal hydrolase 26
cgctatagcttgaatgagtttcaagagaactcattcaagctatagcg

SEQ ID NO: 148 - DNA, 47 bp, Artificial Sequence
  Description of Artificial Sequence DNA encoding an shRNA directed against the gene for the ubiquitin carboxyl- terminal hydrolase 26
gatatcctggctccacacattcaagagatgtgtggagccaggatatc

SEQ ID NO: 149 - DNA, 47 bp, Artificial Sequence
  Description of Artificial Sequence DNA encoding an shRNA directed against the KIAA1097 gene
gagccagtcggatgtagatttcaagagaatctacatccgactggctc

SEQ ID NO: 150 - DNA, 47 bp, Artificial Sequence
  Description of Artificial Sequence DNA encoding an shRNA directed against the KIAA1097 gene
gtaaattctgaaggcgaatttcaagagaattcgccttcagaatttac

SEQ ID NO: 151 - DNA, 47 bp, Artificial Sequence
  Description of Artificial Sequence DNA encoding an shRNA directed against the KIAA1097 gene
gccctcctaaatcaggcaattcaagagattgcctgatttaggagggc

SEQ ID NO: 152 - DNA, 47 bp, Artificial Sequence
  Description of Artificial Sequence DNA encoding an shRNA directed against the KIAA1097 gene
gttgagaaatggagtgaagttcaagagacttcactccatttctcaac

SEQ ID NO: 153 - DNA, 47 bp, Artificial Sequence
  Description of Artificial Sequence DNA encoding an shRNA directed against the gene for the ubiquitin specific protease (USP22) gene
gcttggaaaatgcaaggcgttcaagagacgccttgcattttccaagc

SEQ ID NO: 154 - DNA, 47 bp, Artificial Sequence
  Description of Artificial Sequence DNA encoding an shRNA directed against the gene for the ubiquitin specific protease (USP22) gene
ctgcatcatagaccagatcttcaagagagatctggtctatgatgcag

SEQ ID NO: 155 - DNA, 47 bp, Artificial Sequence
  Description of Artificial Sequence DNA encoding an shRNA directed against the gene for the ubiquitin specific protease (USP22) gene
gatcaccacgtatgtgtccttcaagagaggacacatacgtggtgatc

SEQ ID NO: 156 - DNA, 47 bp, Artificial Sequence
  Description of Artificial Sequence DNA encoding an shRNA directed against the gene for the ubiquitin specific protease 22 (USP22) gene
tgacaacaagtattccctgttcaagagacagggaatacttgttgtca

SEQ ID NO: 157 - DNA, 47 bp, Artificial Sequence
  Description of Artificial Sequence DNA encoding an shRNA directed against the gene for the ubiquitin specific processing protease 29
gaaatataagacagattccttcaagagaggaatctgtcttatatttc

SEQ ID NO: 158 - DNA, 47 bp, Artificial Sequence
  Description of Artificial Sequence DNA encoding an shRNA directed against the gene for the ubiquitin specific processing protease 29
cccatcaagtttagaggatttcaagagaatcctctaaacttgatggg

SEQ ID NO: 159 - DNA, 47 bp, Artificial Sequence
  Description of Artificial Sequence DNA encoding an shRNA directed against the gene for the ubiquitin specific processing protease 29
ggtgtcccatgggaatatattcaagagatatattcccatgggacacc

SEQ ID NO: 160 - DNA, 47 bp, Artificial Sequence
  Description of Artificial Sequence DNA encoding an shRNA directed against the gene for the ubiquitin specific processing protease 29
gaatgccgacctacaaagattcaagagatctttgtaggtcggcattc

SEQ ID NO: 161 - DNA, 47 bp, Artificial Sequence
  Description of Artificial Sequence DNA encoding an shRNA directed against the CYLD gene
cagttatattctgtgatgtttcaagagaacatcacagaatataactg

SEQ ID NO: 162 - DNA, 47 bp, Artificial Sequence
  Description of Artificial Sequence DNA encoding an shRNA directed against the CYLD gene
gaggtgttggggacaaaggttcaagagacctttgtccccaacacctc

SEQ ID NO: 163 - DNA, 47 bp, Artificial Sequence
  Description of Artificial Sequence DNA encoding an shRNA directed against the CYLD gene
gtgggctcattggctgaagttcaagagacttcagccaatgagcccac

SEQ ID NO: 164 - DNA, 47 bp, Artificial Sequence
  Description of Artificial Sequence DNA encoding an shRNA directed against the CYLD gene
gagctactgaggacagaaattcaagagatttctgtcctcagtagctc

SEQ ID NO: 165 - DNA, 47 bp, Artificial Sequence
  Description of Artificial Sequence DNA encoding an shRNA directed against the gene for the ubiquitin carboxyl-terminal hydrolase 2
tcagcaggatgctcaggagttcaagagactcctgagcatcctgctga

SEQ ID NO: 166 - DNA, 47 bp, Artificial Sequence
  Description of Artificial Sequence DNA encoding an shRNA directed against the gene for  the ubiquitin carboxyl-terminal hydrolase 2
gaagttctccatccagaggttcaagagacctctggatggagaacttc

SEQ ID NO: 167 - DNA, 47 bp, Artificial Sequence
  Description of Artificial Sequence DNA encoding an shRNA directed against the gene for the ubiquitin carboxyl-terminal hydrolase 2
gccggtccccaccagcagcttcaagagagctgctggtggggaccggc

SEQ ID NO: 168 - DNA, 47 bp, Artificial Sequence
  Description of Artificial Sequence DNA encoding an shRNA directed against the gene for the ubiquitin carboxyl-terminal hydrolase 2
cactcgggagttgagagatttcaagagaatctctcaactcccgagtg

SEQ ID NO: 169 - DNA, 47 bp, Artificial Sequence
  Description of Artificial Sequence DNA encoding an shRNA directed against the gene for the ubiquitin specific protease 3 (USP3)
gcccttgggtctgtttgacttcaagagagtcaaacagacccaagggc

SEQ ID NO: 170 - DNA, 47 bp, Artificial Sequence
  Description of Artificial Sequence DNA encoding an shRNA directed against the gene for the ubiquitin specific protease 3 (USP3)
ctcaacactaaacagcaagttcaagagacttgctgtttagtgttgag

SEQ ID NO: 171 - DNA, 47 bp, Artificial Sequence
  Description of Artificial Sequence DNA encoding an shRNA directed against the gene for the ubiquitin specific protease 3 (USP3)
gatttcattggacagcatattcaagagatatgctgtccaatgaaatc

SEQ ID NO: 172 - DNA, 47 bp, Artificial Sequence
  Description of Artificial Sequence DNA encoding an shRNA directed against the gene for the ubiquitin specific protease 3 (USP3)
catggggcaccaactaattttcaagagaaattagttggtgccccatg

SEQ ID NO: 173 - DNA, 47 bp, Artificial Sequence
  Description of Artificial Sequence DNA encoding an shRNA directed against the gene for the ubiquitin carboxyl-terminal hydrolase 23
ggtgtctctgcgggattgtttcaagagaacaatcccgcagagacacc

SEQ ID NO: 174 - DNA, 47 bp, Artificial Sequence
  Description of Artificial Sequence DNA encoding an shRNA directed against the gene for the ubiquitin carboxyl-terminal hydrolase 23
agttcagtaggtgtagactttcaagagaagtctacacctactgaact

SEQ ID NO: 175 - DNA, 47 bp, Artificial Sequence
  Description of Artificial Sequence DNA encoding an shRNA directed against the gene for the ubiquitin carboxyl-terminal hydrolase 23
gagttcctgaagctcctcattcaagagatgaggagcttcaggaactc

SEQ ID NO: 176 - DNA, 47 bp, Artificial Sequence
  Description of Artificial Sequence DNA encoding an shRNA directed against the gene for the ubiquitin carboxyl-terminal hydrolase 23
ggatttgctgggggcaaggttcaagagaccttgcccccagcaaatcc

SEQ ID NO: 177 - DNA, 47 bp, Artificial Sequence
  Description of Artificial Sequence DNA encoding an shRNA directed against the UBP-32.7 gene
ctcagaaagccaacattcattcaagagatgaatgttggctttctgag

SEQ ID NO: 178 - DNA, 47 bp, Artificial Sequence
  Description of Artificial Sequence DNA encoding an shRNA directed against the UBP-32.7 gene
cgcattgtaataagaaggtttcaagagaaccttcttattacaatgcg

SEQ ID NO: 179 - DNA, 47 bp, Artificial Sequence
  Description of Artificial Sequence DNA encoding an shRNA directed against the UBP-32.7 gene
gggaggaaaatgcagaaatttcaagagaatttctgcattttcctccc

SEQ ID NO: 180 - DNA, 47 bp, Artificial Sequence
  Description of Artificial Sequence DNA encoding an shRNA directed against the UBP-32.7 gene
ttacaaatttaggaaatacttcaagagagtatttcctaaatttgtaa

SEQ ID NO: 181 - DNA, 47 bp, Artificial Sequence
  Description of Artificial Sequence DNA encoding an shRNA directed against the gene for the Homo sapiens ubiquitin specific protease 13 (isopeptidase T-3)
gttatgaattgatatgcagttcaagagactgcatatcaattcataac

SEQ ID NO: 182 - DNA, 47 bp, Artificial Sequence
  Description of Artificial Sequence DNA encoding an shRNA directed against the gene for the Homo sapiens ubiquitin specific protease 13 (isopeptidase T-3)
gtgataacacaactaatggttcaagagaccattagttgtgttatcac

SEQ ID NO: 183 - DNA, 47 bp, Artificial Sequence
  Description of Artificial Sequence DNA encoding an shRNA directed against the gene for the Homo sapiens ubiquitin specific protease 13 (isopeptidase T-3)
gtagaggagagttctgaaattcaagagatttcagaactctcctctac

SEQ ID NO: 184 - DNA, 47 bp, Artificial Sequence
  Description of Artificial Sequence DNA encoding an shRNA directed against the gene for the Homo sapiens ubiquitin specific protease 13 (isopeptidase T-3)
gcctctaatcctgataaggttcaagagaccttatcaggattagaggc

SEQ ID NO: 185 - DNA, 47 bp, Artificial Sequence
  Description of Artificial Sequence DNA encoding an shRNA directed against the gene for the ubiquitin carboxyl-terminal hydrolase 28
gatgatcttcaggctgccattcaagagatggcagcctgaagatcatc

SEQ ID NO: 186 - DNA, 47 bp, Artificial Sequence
  Description of Artificial Sequence DNA encoding an shRNA directed against the gene for the ubiquitin carboxyl-terminal hydrolase 28
gtatggacaagagcgttggttcaagagaccaacgctcttgtccatac

SEQ ID NO: 187 - DNA, 47 bp, Artificial Sequence
  Description of Artificial Sequence DNA encoding an shRNA directed against the gene for the ubiquitin carboxyl-terminal hydrolase 28
cgaacccttctggaacagtttcaagagaactgttccagaagggttcg

SEQ ID NO: 188 - DNA, 47 bp, Artificial Sequence
  Description of Artificial Sequence DNA encoding an shRNA directed against the gene for the ubiquitin carboxyl-terminal hydrolase 28
gtggcatgaagattatagtttcaagagaactataatcttcatgccac

SEQ ID NO: 189 - DNA, 47 bp, Artificial Sequence
  Description of Artificial Sequence DNA encoding an shRNA directed against the ubiquitin carboxyl-terminal hydrolase 14
ggtgaacaaggacagtatcttcaagagagatactgtccttgttcacc

SEQ ID NO: 190 - DNA, 47 bp, Artificial Sequence
  Description of Artificial Sequence DNA encoding an shRNA directed against the gene for the ubiquitin carboxyl-terminal hydrolase 14
gcaatagaggatgattctgttcaagagacagaatcatcctctattgc

SEQ ID NO: 191 - DNA, 47 bp, Artificial Sequence
  Description of Artificial Sequence DNA encoding an shRNA directed against the gene for the ubiquitin carboxyl-terminal hydrolase 14
tctgtgaatgccaaagttcttcaagagagaactttggcattcacaga

SEQ ID NO: 192 - DNA, 47 bp, Artificial Sequence
  Description of Artificial Sequence DNA encoding an shRNA directed against the gene for the ubiquitin carboxyl-terminal hydrolase 14
cacaccagggaaggtctagttcaagagactagaccttccctggtgtg

SEQ ID NO: 193 - DNA, 47 bp, Artificial Sequence
  Description of Artificial Sequence DNA encoding an shRNA directed against the DUB1 gene
gcaggaagatgcccatgaattcaagagattcatgggcatcttcctgc

SEQ ID NO: 194 - DNA, 47 bp, Artificial Sequence
  Description of Artificial Sequence DNA encoding an shRNA directed agains the DUB1 gene
gaatgtgcaatatcctgagttcaagagactcaggatattgcacattc

SEQ ID NO: 195 - DNA, 47 bp, Artificial Sequence
  Description of Artificial Sequence DNA encoding an shRNA directed against the DUB1 gene
tggatgatgccaaggtcacttcaagagagtgaccttggcatcatcca

SEQ ID NO: 196 - DNA, 47 bp, Artificial Sequence
  Description of Artificial Sequence DNA encoding an shRNA directed against the DUB1 gene
gctccgtgctaaacctctcttcaagagagagaggtttagcacggagc

SEQ ID NO: 197 - DNA, 47 bp, Artificial Sequence
  Description of Artificial Sequence DNA encoding an shRNA directed against the gene for the mouse USP27 homolog
gcctccacctcaacagaggttcaagagacctctgttgaggtggaggc

SEQ ID NO: 198 - DNA, 47 bp, Artificial Sequence
  Description of Artificial Sequence DNA encoding an shRNA directed against the gene for the mouse USP27 homolog
ctgcatcatagaccaaatcttcaagagagatttggtctatgatgcag

SEQ ID NO: 199 - DNA, 47 bp, Artificial Sequence
  Description of Artificial Sequence DNA encoding an shRNA directed against the gene for the mouse USP27 homolog
gatcactacatacatttccttcaagagaggaaatgtatgtagtgatc

SEQ ID NO: 200 - DNA, 47 bp, Artificial Sequence
  Description of Artificial Sequence DNA encoding an shRNA directed against the gene for the mouse USP27 homolog
gtaaagagagcagaatgaattcaagagattcattctgctctctttac

SEQ ID NO: 201 - DNA, 47 bp, Artificial Sequence
  Description of Artificial Sequence DNA encoding an shRNA directed against the gene for the ubiquitin carboxyl-terminal hydrolase 4
cgcggggcgcagtggtatcttcaagagagataccactgcgccccgcg

SEQ ID NO: 202 - DNA, 47 bp, Artificial Sequence
  Description of Artificial Sequence DNA encoding an shRNA directed against the gene for the ubiquitin carboxyl-terminal hydrolase 4
cagaaggcagtggggaagattcaagagatcttccccactgccttctg

SEQ ID NO: 203 - DNA, 47 bp, Artificial Sequence
  Description of Artificial Sequence DNA encoding an shRNA directed against the gene for the ubiquitin carboxyl-terminal hydrolase 4
gcctgggagaatcacaggtttcaagagaacctgtgattctcccaggc

SEQ ID NO: 204 - DNA, 47 bp, Artificial Sequence
  Description of Artificial Sequence DNA encoding an shRNA directed against the gene for the ubiquitin carboxyl-terminal hydrolase 4
accagacaaggaaatacccttcaagagagggtatttccttgtctggt

SEQ ID NO: 205 - DNA, 47 bp, Artificial Sequence
  Description of Artificial Sequence DNA encoding an shRNA directed against the TRE-2 gene
cacatccaccacatcgaccttcaagagaggtcgatgtggtggatgtg

SEQ ID NO: 206 - DNA, 47 bp, Artificial Sequence
  Description of Artificial Sequence DNA encoding an shRNA directed against the TRE-2 gene
gtcacaacccaagaccatgttcaagagacatggtcttgggttgtgac

SEQ ID NO: 207 - DNA, 47 bp, Artificial Sequence
  Description of Artificial Sequence DNA encoding an shRNA directed against the TRE-2 gene
ctcaacaggacaaatcccattcaagagatgggatttgtcctgttgag

SEQ ID NO: 208 - DNA, 47 bp, Artificial Sequence
  Description of Artificial Sequence DNA encoding an shRNA directed against the TRE-2 gene
tagatcaattattgtggatttcaagagaatccacaataattgatcta

SEQ ID NO: 209 - DNA, 47 bp, Artificial Sequence
  Description of Artificial Sequence DNA encoding an shRNA directed against the gene for the ubiquitin carboxyl-terminal hydrolase 15 (UNPH-2)
ggaacaccttattgatgaattcaagagattcatcaataaggtgttcc

SEQ ID NO: 210 - DNA, 47 bp, Artificial Sequence
  Description of Artificial Sequence DNA encoding an shRNA directed against the gene for the ubiquitin carboxyl-terminal hydrolase 15 (UNPH-2)
ctttaacagaaattgtctcttcaagagagagacaatttctgttaaag

SEQ ID NO: 211 - DNA, 47 bp, Artificial Sequence
  Description of Artificial Sequence DNA encoding an shRNA directed agaainst the gene for the ubiquitin carboxyl-terminal hydrolase 15 (UNPH-2)
cctatgcagtacaaagtggttcaagagaccactttgtactgcatagg

SEQ ID NO: 212 - DNA, 47 bp, Artificial Sequence
  Description of Artificial Sequence DNA encoding an shRNA directed against the gene for the ubiquitin carboxyl-terminal hydrolase 15 (UNPH-2)
gatcttttcttgctttggattcaagagatccaaagcaagaaaagatc

SEQ ID NO: 213 - DNA, 47 bp, Artificial Sequence
  Description of Artificial Sequence DNA encoding an shRNA directed against the KIAA1372 gene
cagcatccttcaggccttattcaagagataaggcctgaaggatgctg

SEQ ID NO: 214 - DNA, 47 bp, Artificial Sequence
  Description of Artificial Sequence DNA encoding an shRNA directed against the KIAA1372 gene
gatagtgactcggatctgcttcaagagagcagatccgagtcactatc

SEQ ID NO: 215 - DNA, 47 bp, Artificial Sequence
  Description of Artificial Sequence DNA encoding an shRNA directed against the KIAA1372 gene
gacatcacagcccgggagtttcaagagaactcccgggctgtgatgtc

SEQ ID NO: 216 - DNA, 47 bp, Artificial Sequence
  Description of Artificial Sequence DNA encoding an shRNA directed against the KIAA1372 gene
ggacacagcctatgtgctgttcaagagacagcacataggctgtgtcc

SEQ ID NO: 217 - DNA, 47 bp, Artificial Sequence
  Description of Artificial Sequence DNA encoding an shRNA directed against the gene for the BRCA1 associated protein-1
gtggaggagatctacgaccttcaagagaggtcgtagatctcctccac

SEQ ID NO: 218 - DNA, 47 bp, Artificial Sequence
  Description of Artificial Sequence DNA encoding an shRNA directed against the gene for the BRCA1 associated protein-1
ctcttgtgcaactcatgccttcaagagaggcatgagttgcacaagag

SEQ ID NO: 219 - DNA, 47 bp, Artificial Sequence
  Description of Artificial Sequence DNA encoding an shRNA directed against the gene for the BRCA1 associated protein-1
acagggcccctgcagcctcttcaagagagaggctgcaggggccctgt

SEQ ID NO: 220 - DNA, 47 bp, Artificial Sequence
  Description of Artificial Sequence DNA encoding an shRNA directed against the gene for the BRCA1 associated protein-1
gaagacctggcggcaggtgttcaagagacacctgccgccaggtcttc

(19) United States
(12) Patent Application Publication
(10) Pub. No.: US 2005/0071893 A1
(43) Pub. Date: Mar. 31, 2005

(54) SiRNA mediated gene silencing in transgenic animals

(76) Inventors: Jost Seibler, Koln (DE); Frieder Schwenk, Koln (DE); Ralf Kuhn, Koln (DE); Birgit Kuter-Luks, Cologn (DE)

Correspondence Address: NORRIS, MCLAUGHLIN & MARCUS, PA, 875 THIRD STREET, 18TH FLOOR, NEW YORK, NY, 10022, US

(21) Appl. No.: 10/685,837

(22) Filed: Oct. 15, 2003

Related U.S. Application Data
(60) Provisional application No. 60/420,476, filed on Oct. 22, 2002.
(60) Provisional application No. 60/467,814, filed on May 2, 2003.
(60) Provisional application No. 60/485,969, filed on Jul. 10, 2003.

(30) Foreign Application Priority Data
Oct. 17, 2002 (EP) 02023283.1

(51) Int. Cl. A01K 67/027; C12N 15/85

(52) U.S. Cl. 800/14; 435/455

(57) ABSTRACT

The present invention relates to a process that enables constitutive and inducible gene knock down in living organisms using a short hairpin RNA expression vector integrated into the genome of the organism.

The present invention relates to a process that enables constitutive and inducible gene knock down in living organisms using a short hairpin RNA expression vector integrated into the genome of the organism.

### BACKGROUND OF THE INVENTION

RNA interference (RNAi) has been discovered some years ago as a tool for inhibition of gene expression (Fire, A. et al., Nature 391, 806-811 (1998)). It based on the introduction of double stranded RNA (dsRNA) molecules into cells, whereby one strand is complementary to the coding region of a target gene. Through pairing of the specific mRNA with the introduced RNA molecule, the mRNA is degraded by a cellular mechanism. Since long dsRNA provokes an interferon response in mammalian cells, the technology was initially restricted to organisms or cells showing no interferon response (Bass, B. L., Nature 411, 428-429 (2001)). The finding that short (<30 bp) interfering RNAs (siRNA) circumvent the interferon response extended the application to mammalian cells (Elbashir, S. M. et al., Nature 411, 494-498 (2001)).

Although RNAi in mice has been in principle demonstrated, the current technology does not allow performing systematic gene function analysis in vivo. So far the inhibition of gene expression has been achieved by injection of purified siRNA into the tail vain of mice (McCaffrey, A. P. et al., Nature 418, 38-39 (2002); Lewis, D. H. et al., Nature Genet. 32, 107-108 (2002)). Using this approach, gene inhibition is restricted to specific organs and persists only a few days. A further improvement of the siRNA technology is based on the intracellular transcription of short hairpin RNA (shRNA) molecules using gene expression vectors (see FIG. 1; Brummelkamp, T. R. et al., Science 296, 550-553 (2002); Paddison, P. J. et al, Genes Dev. 16, 948-958 (2002); Yu, J. Y. et al., Proc. Natl. Acad. Sci. USA 99, 6047-6052 (2002); Sui, G. et al., Proc. Natl. Acad. Sci. USA 99, 5515-5520 (2002); Paul, C. P. et al., Nature Biotechnol. 20, 505-508 (2002); Xia, H. et al., Nat. Biotechnol. 10, 1006-10 (2002); Jacque, J. M. et al., Nature 418(6896):435-8 (2002)). The activity of shRNA in mice has been demonstrated by McCaffrey et al., 2002 through injection of shRNA expression vectors into the tail vain. Again, gene inhibition was temporally and spatially restricted. Although these results demonstrate that the mechanism of shRNA mediated gene silencing is functional in mice, they do not clarify whether constitutive RNAi can be achieved in transgenic animals. Brummelkamp, T. R. et al., Science 296, 550-553 (2002), Paddison, P. J. et al., Genes Dev. 16, 948-958 (2002), Hemann, M. T. et al., Nat. Genet. 33(3):396-400 (2003); and Devroe, E. et al., BMC Biotechnol. 2(1):15 (2002) have shown the long-term inhibition of gene expression through stable integration of shRNA vectors in cultivated cell lines. These experiments included random integration of shRNA transgenes and screening for clones with appropriate siRNA expression, which is not applicable for testing of a large number of different shRNA transgenes in mice. Finally, several reports have demonstrated shRNA-mediated gene silencing in transgenic mice and rats (Hasuwa, H. et al., FEBS Lett. 532(1-2):227-30 (2002); Carmell, M. A. et al., Nat. Struct. Biol. 10(2):91-2 (2003); Rubinson, D. A. et al., Nat. Genet. 33(3):401-6 (2003); Kunath, T. et al., Nat. Biotechnol. (Apr. 7, 2003)). However, these experiments again included random integration of shRNA transgenes resulting in variable levels and patterns of shRNA expression. Thus, testing of ES cell clones or mouse lines with appropriate shRNA expression had been required, which is a laborious and time-consuming undertaking.

The in vivo validation of genes by RNAi mediated gene repression in a large scale setting requires the expression of siRNA at sufficiently high levels and with a predictable pattern in multiple organs. Targeted transgenesis provides the only approach to achieve reproducible expression of transgenes in the living organism (e.g. mammalians such as mice). It has been, however, questionable whether a single copy of a siRNA expression vector integrated into the genome would result in sufficiently high levels of siRNA required for RNAi-mediated gene inhibition in multiple organs of the living organism.

Most siRNA expression vectors are based on polymerase III dependent (Pol III) promoters (U6 or H1) that allow the production of transcripts carrying only a few non-homologous bases at their 3′ ends. It has been shown that the presence of non-homologous RNA at the ends of the shRNA stretches lower the efficiency of RNAi mediated gene silencing (Xia et al., Nat. Biotechnol. 10, 1006-10 (2002)). However, it has been unpredictable for a person skilled in the art which genomic region within the living organism promotes an appropriate expression pattern of the Pol III promoter driven shRNA constructs required for RNAi-mediated gene inhibition in multiple organs of the living organism.

For the temporal control of RNAi mediated gene silencing in transgenic cells lines and living organism, a tight system for inducible siRNA expression is needed. Inducible gene expression systems based on the tetracycline dependent repressor are known. It is, however, not obvious whether a stably integrated, single copy configuration of these systems can be created that allows inducible RNAi in multiple organs without background activity.

### SUMMARY OF THE INVENTION

It has now been found that the modification of murine embryonic stem (ES) cells by the introduction of shRNA expression vectors through targeted transgenesis allows the inhibition of gene expression in mice generated from these modified ES cells. The invention thus provides
(1) the use of an expression vector comprising a short hairpin RNA (shRNA) construct under control of a ubiquitous promoter for preparing an agent for constitutive and/or inducible gene knock down (hereinafter also referred to as “RNAi mediated inhibition of gene expression”) in a living organism, in tissue culture or in cells of a cell culture;
(2) a method for constitutive and/or inducible gene knock down in a living organism, in tissue culture or in cells of a cell culture which comprises stably integrating an expression vector as defined in (1) above into the genome of the living organism, in the genome of the tissue culture or into the genome of the cells of the cell culture;
(3) a living organism, tissue culture or cell culture having stably integrated, preferably at a polymerase II dependent locus of the living organism, tissue culture or cells of the cell culture, an expression vector as defined in (1) above; and
(4) an expression vector as defined in (1) above.

The agent of embodiment (1) is suitable for in vivo and in vitro applications.

The living organisms in embodiments (1) to (3) above are preferably vertebrates. In embodiments (2) and (3) the living organisms are more preferably non-human vertebrates. The tissue cultures and the cells of the cell cultures of (1) to (3) above are preferably derived from vertebrates. The method of embodiment (2) includes in vivo and in vitro reaction schemes.

### SHORT DESCRIPTION OF THE FIGURES

FIG. 1: Mechanism of RNAi through shRNA expression. A: expression of shRNA. B: shRNA processing. C: Pairing of siRNA with complementary mRNA strands. D: Cleavage of mRNA.

FIG. 2: Pol III dependent shRNA expression vector (constitutive). Insertion of an shRNA expression vector into a ubiquitously expressed genomic locus. Transcription through the upstream Pol II dependent promoter will be stopped by a synthetic polyadenylation signal (pA) and the hGH pA. The Pol III dependent promoter controls the expression of shRNA. The transcript is stopped by five thymidine bases. The exact sequence of suitable target vectors is given in SEQ ID Nos:6 and 8.

FIG. 3: Tet repression system. tTS is a doxycycline controlled transcriptional silencer consisting of the tet repressor and the KRAB-AB domain of the Kid-1 protein. This fusion protein binds to the pol III dependent promoter via the tet operator sequences only in the absence of doxycycline and represses transcription.

FIG. 4: Vectors for Pol III dependent promoter based tet-repression system (inducible). Insertion of a shRNA expression vector into a ubiquitous expressed genomic locus. The transcription of the Pol II dependent promoter will be stopped by the synthetic polyadenylation signal (pA) and a hGH pA. An inducible Pol III dependent promoter controls the expression of shRNA. The transcript is stopped by five thymidine bases.

FIG. 5: Principle of the Doxycycline inducible gene expression system. rtTA2^{M2} is a doxycycline-controlled transcriptional activator consisting of the reverse tet repressor and the VP16 activation domain of the Herpes simplex virus VP16 protein. This fusion protein binds to a minimal CMV-promoter via the tet operator sequences in the presence of doxycycline and activates transcription. tTS functions in a similar fashion as explained in FIG. 3.

FIG. 6: Vectors for Pol II dependent promoter based tet-system (inducible). Insertion of a shRNA expression vector into a ubiquitous expressed genomic locus. The rtTA2^{M2} is expressed under the control of the ubiquitous Pol II dependent promoter, stopped by a synthetic polyadenylation signal (pA) and a hGH pA. An inducible Pol II dependent promoter controls the expression of shRNA. The transcript is stopped by a short polyadenylation signal.

FIG. 7: ShRNA-mediated inhibition of luciferase gene expression in embryonic stem cells. All cells carried stably integrated expression cassettes of firefly- (Fluc) and Renilla luciferases (Rluc). Simultaneous expression of a Fluc-specific shRNA under control of the H1- (H1-shRNA) or U6-promoter (U6-shRNA) resulted in a ˜75% and ˜60% reduction of firefly luciferase activity, respectively. The values of Fluc activity were normalized by using the Rluc activity for reference.

FIG. 8: Vectors for a U6 promoter based tet-repression system (inducible). Transcription of the Pol II promoter will be stopped by the synthetic polyadenylation signal (pA) and a hGH pA sequence. The inducible U6 promoter controls expression of the Fluc-specific shRNA. Transcription of the shRNA is stopped by five thymidine bases. TetR is a doxycycline controlled repressor (Gossen and Bujard, PNAS. 89: 5547-5551 (1992)) expressed by the CAGGS-promoter. The tetR binds to the pol III promoter via the tet operator sequences only in the absence of doxycycline and blocks transcription.

FIG. 9: Vectors for H1 promoter based tet-repression system (inducible). The inducible H1 promoter controls the expression of shRNA. TetR is a doxycycline controlled repressor expressed by the CAGGS-promoter. The tetR binds to the pol III promoter via the tet operator sequences only in the absence of doxycycline and blocks transcription.

FIG. 10: Inducible RNA interference in embryonic stem cells using tetracycline dependent U6 promoters. All cells carried stably integrated expression cassettes of firefly luciferase (Fluc), Renilla luciferase (Rluc), and the tet repressor. Doxycycline-inducible expression of a Fluc-specific shRNA under control of the U6_{tetO1}- (Art4.12 RNAi 23/36 C2) or U6_{tetO2}-promoter (Art4.12 RNAi 24/36 A-D11) resulted in a ˜30% and ˜50% reduction of firefly luciferase activity, respectively. The values of Fluc activity were normalized by using the Rluc activity for reference (FIG. 12).

FIG. 11: Inducible RNA interference using tetracycline dependent H1 promoters NIH3T3. Cells were transiently transfected with firefly luciferase (Fluc), tet repressor, LacZ, and shRNA expression vectors. Doxycycline-inducible expression of the Fluc-specific shRNA under control of the H1_{tetO2/5′}-, H1_{tetO2/3′}-, or H1_{tetO2/5′+3′}-promoter resulted in a ˜40%, ˜80%, and ˜40% reduction of firefly luciferase activity, respectively. The values of Fluc activity were normalized by using the LacZ activity for reference.

FIG. 12: Experimental strategy. Genes of the firefly and Renilla luciferases are expressed through the endogenous rosa26 promoter via splice acceptor sites. Specific shRNA mediated repression of the firefly luciferase gene can be measured by using the Renilla luciferase activity as a reference.

FIG. 13: ShRNA-mediated inhibition of luciferase expression in mice.

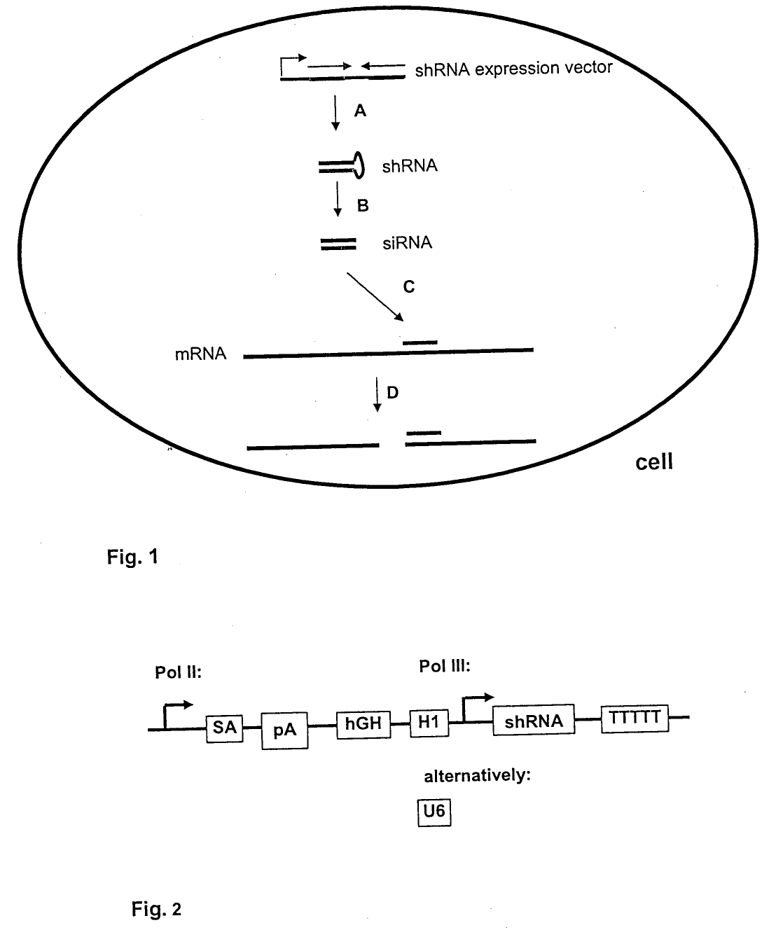

A: Firefly luciferase activity in mice in the absence (delta shFluc, black bars) or presence of the U6- (U6-shFluc, white bars) and H1-shRNA transgenes (H1-shFluc, grey bars), respectively. All mice carried the firefly and the Renilla luciferase transgenes. Relative values of Firefly luciferase activitiy in different organs are given as indicated. All values of Fluc activity were normalized by using the Rluc activity for reference.

B: Efficiency of shRNA-mediated repression of firefly luciferase expression in mice. Percentages of U6- (white bars) and H1-shRNA mediated (grey bars) repression of Firefly luciferase activity are shown. ND: not determined.

### DETAILED DESCRIPTION OF THE INVENTION

The term “living organisms” according to the present invention relates to multi-cell organisms which can be living vertebrates (hereinafter shortly referred to as “vertrebrates”) such as mammals (e.g. non-human animals such as rodents (including mice and rats, etc.; and humans) or non-mammals (e.g. fish) or can be invertebrates such as insects or worms, or can be plants (higher plants, algi or fungi). Most preferred living organisms are vertebrates, in particular mice and fish.

“Tissue culture” according to the present invention refers to parts of the above defined living organisms (including organs and the like) which are cultured in vitro.

“Cell culture” according to the present invention includes cells isolated from the above defined living organisms which are cultured in vitro. These cells can be transformed (immortalized) or untransformed (directly derived from living organisms; primary cell culture).

The expression vector according to the invention of the present application is suitable for stable integration into the living organism or into cells of the cell culture (vectors for transient integration are also contemplated). It is moreover preferred that it contains homologous sequences suitable for targeted integration at a defined locus, preferably at a polymerase II dependent locus of the living organisms or cells of the cell culture. Such polymerase II dependent loci include, but are not limited to, Rosa26 locus (the murine Rosa26 locus being depicted in SEQ ID NO:1), collagen, RNA polymerase, actin and HPRT.

The expression vector may further contain functional sequences selected from splice acceptor sequences (such as a splice acceptor of adenovirus, etc.), polyadenylation sites (such as synthetic polyadenylation sites, the polyadenylation site of human growth hormones, or the like), selectable marker sequences (such as the neomycin phosphotransferase gene of E. coli transposon, etc.), etc.

The ubiquitous promoter in the vector according to the invention is preferably selected from polymerase I, II and III dependent promoters, preferably is a polymerase II or III dependent promoter including, but not limited to, a CMV promoter, a CAGGS promoter, a snRNA promoter such as U6, a RNAse P RNA promoter such as H1, a tRNA promoter, a 7SL RNA promoter, a 5 S rRNA promoter, etc.

The ubiquitous promoter can be a constitutive promoter, or can be an inducible promoter. Suitable inducible promoters are the above-mentioned polymerase I, II and III dependent promoters containing an operator sequence including, but not limited to, tet, Gal4, lac, etc.

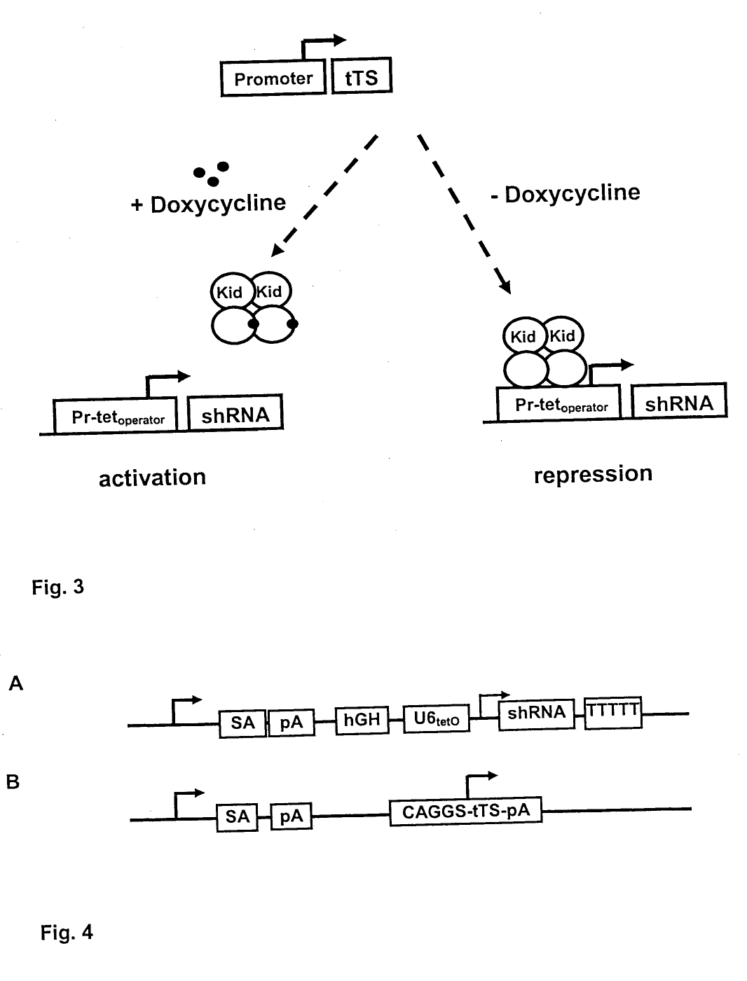

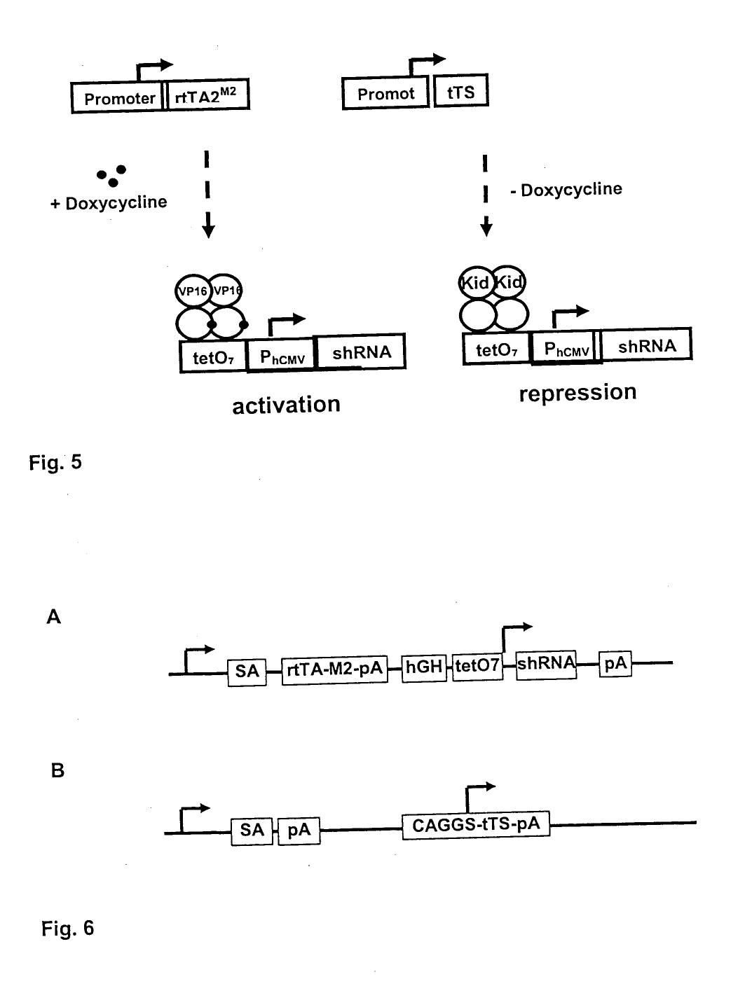

The expression vector of the invention is suitable for the following particularly preferred approaches (for constitutive and inducible expression):
A. a Pol III dependent promoter (constitutive U6, H1 or the like) driven shRNA construct (to be integrated into a ubiquitously active Pol II dependent locus (see FIG. 2);
B. a Pol III dependent promoter (inducible U6, H1 or the like) driven shRNA construct (to be integrated into a ubiquitously active Pol II dependent locus (FIGS. 3 and 4)); or
C. a polymerase II (Pol II) dependent promoter (inducible CMV or the like) driven shRNA construct (to be integrated into a ubiquitously active Pol II dependent locus (FIGS. 5 and 6)).

The ShRNA within the vector of the invention preferably comprises
(I) at least one DNA segment A-B-C wherein

A is a 15 to 35, preferably 19 to 29 bp DNA sequence being at least 95%, preferably 100% complementary to the gene to be knocked down (e.g. firefly luciferase, p53, etc.);

B is a spacer DNA sequence having 5 to 9 bp forming the loop of the expressed RNA hair pin molecule, and

C is a 15 to 35, preferably 19 to 29 bp DNA sequence being at least 85% complementary to the sequence A; and
(II) a stop and/or polyadenylation sequence.

Suitable shRNA sequences for the knock down of a given target gene are well known in the art (see e.g. the particular shRNA sequences mentioned in Tables 1 and 2 below) or can readily be determined by the skilled artesian.

TABLE 1

| | | |
|---|---|---|
| target | | |
| gene | shRNA sequence/SEQ ID NO | Reference |

| | | | |
|---|---|---|---|
| CDH-1 | TgagaagtctcccagtcagTTCAAGAGActgactgggagacttctca | (19) | Brummelkamp |
| p53 | GactccagtggtaatctacTTCAAGAGAgtagattaccactggagtc | (20) | et al., |
| CDC20 | CggcaggactccgggccgaTTCAAGAGAtcggcccggagtcctgccg | (21) | Science, 296: |
| | | | 550-3 (2002). |
| CYLD | CctcatgcagttctctttgTTCAAGAGAcaaagagaactgcatgagg | (22) | Kovalenko et |
| | | | al, Nature, |
| | | | 424: 801-5 |
| | | | (2003). |
| Ras- | AagatgaagccactccctatttCAAGAGAaaatagggagtggcttcatctt | (23) | Kunath et al., |
| Gap | | | Nature |
| | | | Biotechnology, |
| | | | 21: 559-561 |
| | | | (2003). |
| tubulin | GacagagccaagtggactcACAgagtccacttggctctgtc | (24) | Yu et al., |
| | | | PNAS, 99: |
| | | | 6047-52 |
| | | | (2002) |
| lamin | Ctggacttccagaagaacattcgtgttcttctggaagtccag | (25) | Paul et al., |
| | | | Nature Bio- |
| | | | technology, |
| | | | 20: 505-8 |
| | | | (2002). |

TABLE 2

shRNA sequences known from Brummelkamp et al., Nature, 424: 797-
801 (2003):

| | |
|---|---|
| Target | |
| Gene | shRNA Sequence/SEQ ID NO |

| | | |
|---|---|---|
| UBIQUITIN | GAGATTGGTCCAGAACAGTTTCAAGAGAACTGTTCTGGACCAATCTC | (26) |
| CARBOXYL- | GCCCTTCCGATCATGGTAGTTCAAGAGACTACCATGATCGGAAGGGC | (27) |
| TERMINAL | TCTTTAGAATTCTTAAGTATTCAAGAGATACTTAAGAATTCTAAAGA | (28) |
| HYDROLASE 12 | CATTAGCTATATCAACATGTTCAAGAGACATGTTGATATAGCTAATG | (29) |
| UBIQUITIN | ACCACAAACGGCGGAACGATTCAAGAGATCGTTCCGCCGTTTGTGGT | (30) |
| CARBOXYL- | GAGGGTCTTGGAGGTCTTCTTCAAGAGAGAAGACCTCCAAGACCCTC | (31) |
| TERMINAL | GTCCATGCCCAGCCGTACATTCAAGAGATGTACGGCTGGGCATGGAC | (32) |
| HYDROLASE 11 | GCTGGACACCCTCGTGGAGTTCAAGAGACTCCACGAGGGTGTCCAGC | (33) |
| UBIQUITIN | GAATATCAGAGAATTGAGTTTCAAGAGAACTCAATTCTCTGATATTC | (34) |
| CARBOXYL- | TGGACTTCATGAGGAAATGTTCAAGAGACATTTCCTCATGAAGTCCA | (35) |
| TERMINAL | TATTGAATATCCTGTGGACTTCAAGAGAGTCCACAGGATATTCAATA | (36) |
| HYDROLASE 10 | TTGTACTGAGAGAAACTGCTTCAAGAGAGCAGTTTCTCTCAGTACAA | (37) |
| HAUSP | GATCAATGATAGGTTTGAATTCAAGAGATTCAAACCTATCATTGATC | (38) |
| | GGAGTTTGAGAAGTTTAAATTCAAGAGATTTAAACTTCTCAAACTCC | (39) |
| | GAACTCCTCGCTTGCTGAGTTCAAGAGACTCAGCAAGCGAGGAGTTC | (40) |
| | CCGAATTTAACAGAGAGAATTCAAGAGATTCTCTCTGTTAAATTCGG | (41) |
| UBIQUITIN | GACAGCAGAAGAATGCAGATTCAAGAGATCTGCATTCTTCTGCTGTC | (42) |
| CARBOXYL- | ATAAAGCTCAACGAGAACCTTCAAGAGAGGTTCTCGTTGAGCTTTAT | (43) |
| TERMINAL | GGTGAAGTGGCAGAAGAATTTCAAGAGAATTCTTCTGCCACTTCACC | (44) |
| HYDROLASE 8 | GTATTGCAGTAATCATCACTTCAAGAGAGTGATGATTACTGCAATAC | (45) |
| FLJ10785 | GATATGGGGTTCCATGTCATTCAAGAGATGACATGGAACCCCATATC | (46) |
| | GGAGACATGGTTCTTAGTGTTCAAGAGACACTAAGAACCATGTCTCC | (47) |
| | AGCACCAAGTTCGTCTCAGTTCAAGAGACTGAGACGAACTTGGTGCT | (48) |
| | GATGCAACACTGAAAGAACTTCAAGAGAGTTCTTTCAGTGTTGCATC | (49) |
| KIAA0710 | GTCAATGGCAGTGATGATATTCAAGAGATATCATCACTGCCATTGAC | (50) |
| | CCTGCTAGCTGCCTGTGGCTTCAAGAGAGCCACAGGCAGCTAGCAGG | (51) |
| | CCACCTTTGCCAGAAGGAGTTCAAGAGACTCCTTCTGGCAAAGGTGG | (52) |
| | CCCTATTGAGGCAAGTGTCTTCAAGAGAGACACTTGCCTCAATAGGG | (53) |
| FLJ12552/ | GAAGGAAAACTTGCTGACGTTCAAGAGACGTCAGCAAGTTTTCCTTC | (54) |
| FLJ14256 | CTCACCTGGGTCCATGAGATTCAAGAGATCTCATGGACCCAGGTGAG | (55) |
| | GCTGTCTTACCGTGTGGTCTTCAAGAGAGACCACACGGTAAGACAGC | (56) |
| | CCTGGACCGCATGTATGACTTCAAGAGAGTCATACATGCGGTCCAGG | (57) |
| KIAA1203 | GTCAATGGCAGTGATGATATTCAAGAGATATCATCACTGCCATTGAC | (58) |
| | CCTGCTAGCTGCCTGTGGCTTCAAGAGAGCCACAGGCAGCTAGCAGG | (59) |
| | CCACCTTTGCCAGAAGGAGTTCAAGAGACTCCTTCTGGCAAAGGTGG | (60) |
| | CCCTATTGAGGCAAGTGTCTTCAAGAGAGACACTTGCCTCAATAGGG | (61) |
| FLJ23277 | GGAAATCCGAATTGCTTGGTTCAAGAGACCAAGCAATTCGGATTTCC | (62) |
| | CACATTTCTTCAAGTGTGGTTCAAGAGACCACACTTGAAGAAATGTG | (63) |
| | CAGCAGGATGCTCAAGAATTTCAAGAGAATTCTTGAGCATCCTGCTG | (64) |
| | GCTGAATACCTACATTGGCTTCAAGAGAGCCAATGTAGGTATTCAGC | (65) |
| FLJ14914 (similar | GGGCTTGTGCCTGGCCTTGTTCAAGAGACAAGGCCAGGCACAAGCCC | (66) |
| to UBP4) | GCCTTGTCCTGCCAAGAAGTTCAAGAGACTTCTTGGCAGGACAAGGC | (67) |
| | GATTGAAGCCAAGGGAACGTTCAAGAGACGTTCCCTTGGCTTCAATC | (68) |
| | TGGCGCCTGCTCCCCATCTTTCAAGAGAAGATGGGGAGCAGGCGCCA | (69) |
| UBIQUITIN CAR- | GAACCAGCAGGCTCTGTGGTTCAAGAGACCACAGAGCCTGCTGGTTC | (70) |
| BOXYL-TERMINAL | GGAAGCATAATTATCTGCCTTCAAGAGAGGCAGATAATTATGCTTCC | (71) |
| HYDROLASE | AGAAGAAGATGCTTTTCACTTCAAGAGAGTGAAAAGCATCTTCTTCT | (72) |
| ISOZYME L5 | CTTGCAGAGGAGGAACCCATTCAAGAGATGGGTTCCTCCTCTGCAAG | (73) |
| UBIQUITIN CAR- | GCAAACAATCAGCAATGCCTTCAAGAGAGGCATTGCTGATTGTTTGC | (74) |
| BOXYL-TERMINAL | TTGGACTGATTCATGCTATTTCAAGAGAATAGCATGAATCAGTCCAA | (75) |
| HYDROLASE | CTGGCAATTCGTTGATGTATTCAAGAGATACATCAACGAATTGCCAG | (76) |
| ISOZYME L3 | TTAGATGGGCGGAAGCCATTTCAAGAGAATGGCTTCCGCCCATCTAA | (77) |
| UBIQUITIN CAR- | GAGGAGTCTCTGGGCTCGGTTCAAGAGACCGAGCCCAGAGACTCCTC | (78) |
| BOXYL-TERMINAL | GAGCTGAAGGGACAAGAAGTTCAAGAGACTTCTTGTCCCTTCAGCTC | (79) |
| HYDROLASE | TGTCGGGTAGATGACAAGGTTCAAGAGACCTTGTCATCTACCCGACA | (80) |
| ISOZYME L1 | CACAGCTGTTCTTCTGTTCTTCAAGAGAGAACAGAAGAACAGCTGTG | (81) |
| KIAA1891/ | GTGGAAGCCTTTACAGATCTTCAAGAGAGATCTGTAAAGGCTTCCAC | (82) |
| FLJ25263 | CAACAGCTGCCTTCATCTGTTCAAGAGACAGATGAAGGCAGCTGTTG | (83) |
| | CCATAGGCAGTCCTCCTAATTCAAGAGATTAGGAGGACTGCCTATGG | (84) |
| | TGTATCACTGCCACTGGTTTTCAAGAGAAACCAGTGGCAGTGATACA | (85) |
| FLJ14528 (similar | CATGTTGGGCAGCTGCAGCTTCAAGAGAGCTGCAGCTGCCCAACATG | (86) |
| to UBP8) | CACAACTGGAGACCTGAAGTTCAAGAGACTTCAGGTCTCCAGTTGTG | (87) |
| | GTATGCCTCCAAGAAAGAGTTCAAGAGACTCTTTCTTGGAGGCATAC | (88) |
| | CTTCACAGTACATTTCTCTTTCAAGAGAAGAGAAATGTACTGTGAAG | (89) |
| U4/U6 TRI SNRNP | GTACTTTCAAGGCCGGGGTTTCAAGAGAACCCCGGCCTTGAAAGTAC | (90) |
| 65 kDa protein | CTTGGACAAGCAAGCCAAATTCAAGAGATTTGGCTTGCTTGTCCAAG | (91) |
| | GACTATTGTGACTGATGTTTTCAAGAGAAACATCAGTCACAATAGTC | (92) |
| | GGAGAACTTTCTGAAGCGCTTCAAGAGAGCGCTTCAGAAAGTTCTCC | (93) |
| XM_089437 | GACGAGAGAAACCTTCACCTTCAAGAGAGGTGAAGGTTTCTCTCGTC | (94) |
| | ACATTATTCTACATTCTTTTTCAAGAGAAAAGAATGTAGAATAATGT | (95) |
| | AGATTCGCAAATGGATGTATTCAAGAGATACATCCATTTGCGAATCT | (96) |
| | CATTCCCACCATGAGTCTGTTCAAGAGACAGACTCATGGTGGGAATG | (97) |
| KIAA1453 | GATCGCCCGACACTTCCGCTTCAAGAGAGCGGAAGTGTCGGGCGATC | (98) |
| | CCAGCAGGCCTACGTGCTGTTCAAGAGACAGCACGTAGGCCTGCTGG | (99) |
| | GCCAGCTCCTCCACAGCACTTCAAGAGAGTGCTGTGGAGGAGCTGGC | (100) |
| | CGCCGCCAAGTGGAGCAGATTCAAGAGATCTGCTCCACTTGGCGGCG | (101) |
| FLJ12697 | GAAGATGCCCATGAATTCCTTCAAGAGAGGAATTCATGGGCATCTTC | (102) |
| | CAAACAGGCTGCGCCAGGCTTCAAGAGAGCCTGGCGCAGCCTGTTTG | (103) |
| | ACGGCCTAGCGCCTGATGGTTCAAGAGACCATCAGGCGCTAGGCCGT | (104) |
| | CTGTAACCTCTCTGATCGGTTCAAGAGACCGATCAGAGAGGTTACAG | (105) |
| UBIQUITIN | TCTGTCAGTCCATCCTGGCTTCAAGAGAGCCAGGATGGACTGACAGA | (106) |
| SPECIFIC | TGAAGCGAGAGTCTTGTGATTCAAGAGATCACAAGACTCTCGCTTCA | (107) |
| PROTEASE 18 | GATGGAGTGCTAATGGAAATTCAAGAGATTTCCATTAGCACTCCATC | (108) |
| (USP18) | CCTTCAGAGATTGACACGCTTCAAGAGAGCGTGTCAATCTCTGAAGG | (109) |
| UBIQUITIN | CCTGACCACGTTCCGACTGTTCAAGAGACAGTCGGAACGTGGTCAGG | (110) |
| CARBOXYL- | GAGTTCCTTCGCTGCCTGATTCAAGAGATCAGGCAGCGAAGGAACTC | (111) |
| TERMINAL | GACTGCCTTGCTGCCTTCTTTCAAGAGAAGAAGGCAGCAAGGCAGTC | (112) |
| HYDROLASE 20 | CGCCGAGGGCTACGTACTCTTCAAGAGAGAGTACGTAGCCCTCGGCG | (113) |
| UBIQUITIN | GGCGAGAAGAAAGGACTGTTTCAAGAGAACAGTCCTTTCTTCTCGCC | (114) |
| CARBOXYL- | GGACGAGAATTGATAAAGATTCAAGAGATCTTTATCAATTCTCGTCC | (115) |
| TERMINAL | GCACGAGAATTTGGGAATCTTCAAGAGAGATTCCCAAATTCTCGTGC | (116) |
| HYDROLASE 24 | CTACTTCATGAAATATTGGTTCAAGAGACCAATATTTCATGAAGTAG | (117) |
| KIAA1594 | GATAACAGCTTCTTGTCTATTCAAGAGATAGACAAGAAGCTGTTATC | (118) |
| | GAGAATAGGACATCAGGGCTTCAAGAGAGCCCTGATGTCCTATTCTC | (119) |
| | CTTGGAAGACTGAACCTGTTTCAAGAGAACAGGTTCAGTCTTCCAAG | (120) |
| | CAACTCCTTTGTGGATGCATTCAAGAGATGCATCCACAAAGGAGTTG | (121) |
| KIAA1350 | GATGTTGTCTCCAAATGCATTCAAGAGATGCATTTGGAGACAACATC | (122) |
| | CGTGGGGACTGTACCTCCCTTCAAGAGAGGGAGGTACAGTCCCCACG | (123) |
| | GTACAGCTTCAGAACCAAGTTCAAGAGACTTGGTTCTGAAGCTGTAC | (124) |
| UBIQUITIN | GATGATCTTCAGAGAGCAATTCAAGAGATTGCTCTCTGAAGATCATC | (125) |
| CARBOXYL- | GGAACATCGGAATTTGCCTTTCAAGAGAAGGCAAATTCCGATGTTCC | (126) |
| TERMINAL | GAGCTAGTGAGGGACTCTTTTCAAGAGAAAGAGTCCCTCACTAGCTC | (127) |
| HYDROLASE 25 | GCAGGGTTCTTTAAGGCAATTCAAGAGATTGCCTTAAAGAACCCTGC | (128) |
| UBIQUITIN | TCGATGATTCCTCTGAAACTTCAAGAGAGTTTCAGAGGAATCATCGA | (129) |
| CARBOXYL- | GATAATGGAAATATTGAACTTCAAGAGAGTTCAATATTTCCATTATC | (130) |
| TERMINAL | GTTCTTCATTTAAATGATATTCAAGAGATATCATTTAAATGAAGAAC | (131) |
| HYDROLASE 16 | GTTAACAAACACATAAAGTTTCAAGAGAACTTTATGTGTTTGTTAAC | (132) |
| USP9X | GTTAGAGAAGATTCTTCGTTTCAAGAGAACGAAGAATCTTCTCTAAC | (133) |
| | GTTGATTGGACAATTAAACTTCAAGAGAGTTTAATTGTCCAATCAAC | (134) |
| | GGTTGATACCGTAAAGCGCTTCAAGAGAGCGCTTTACGGTATCAACC | (135) |
| | GCAATGAAACGTCCAATGGTTCAAGAGACCATTGGACGTTTCATTGC | (136) |
| USP9Y | AGCTAGAGAAAATTCTTCGTTCAAGAGACGAAGAATTTTCTCTAGCT | (137) |
| | GATCCTATATGATGGATGATTCAAGAGATCATCCATCATATAGGATC | (138) |
| | GTTCTTCTTGTCAGTGAAATTCAAGAGATTTCACTGACAAGAAGAAC | (139) |
| | CTTGAGCTTGAGTGACCACTTCAAGAGAGTGGTCACTCAAGCTCAAG | (140) |
| UBIQUITIN | GACCGGCCAGCGAGTCTACTTCAAGAGAGTAGACTCGCTGGCCGGTC | (141) |
| CARBOXYL- | GGACCTGGGCTACATCTACTTCAAGAGAGTAGATGTAGCCCAGGTCC | (142) |
| TERMINAL | CTCTGTGGTCCAGGTGCTCTTCAAGAGAGAGCACCTGGACCACAGAG | (143) |
| HYDROLASE 5 | GACCACACGATTTGCCTCATTCAAGAGATGAGGCAAATCGTGTGGTC | (144) |
| UBIQUITIN | TGGCTTGTTTATTGAAGGATTCAAGAGATCCTTCAATAAACAAGCCA | (145) |
| CARBOXYL- | GTGAATTTGGGGAAGATAATTCAAGAGATTATCTTCCCCAAATTCAC | (146) |
| TERMINAL | CGCTATAGCTTGAATGAGTTTCAAGAGAACTCATTCAAGCTATAGCG | (147) |
| HYDROLASE 26 | GATATCCTGGCTCCACACATTCAAGAGATGTGTGGAGCCAGGATATC | (148) |
| KIAA1097 | GAGCCAGTCGGATGTAGATTTCAAGAGAATCTACATCCGACTGGCTC | (149) |
| | GTAAATTCTGAAGGCGAATTTCAAGAGAATTCGCCTTCAGAATTTAC | (150) |
| | GCCCTCCTAAATCAGGCAATTCAAGAGATTGCCTGATTTAGGAGGGC | (151) |
| | GTTGAGAAATGGAGTGAAGTTCAAGAGACTTCACTCCATTTCTCAAC | (152) |
| UBIQUITIN | GCTTGGAAAATGCAAGGCGTTCAAGAGACGCCTTGCATTTTCCAAGC | (153) |
| SPECIFIC | CTGCATCATAGACCAGATCTTCAAGAGAGATCTGGTCTATGATGCAG | (154) |
| PROTEASE 22 | GATCACCACGTATGTGTCCTTCAAGAGAGGACACATACGTGGTGATC | (155) |
| (USP22) | TGACAACAAGTATTCCCTGTTCAAGAGACAGGGAATACTTGTTGTCA | (156) |
| UBIQUITIN- | GAAATATAAGACAGATTCCTTCAAGAGAGGAATCTGTCTTATATTTC | (157) |
| SPECIFIC | CCCATCAAGTTTAGAGGATTTCAAGAGAATCCTCTAAACTTGATGGG | (158) |
| PROCESSING | GGTGTCCCATGGGAATATATTCAAGAGATATATTCCCATGGGACACC | (159) |
| PROTEASE 29 | GAATGCCGACCTACAAAGATTCAAGAGATCTTTGTAGGTCGGCATTC | (160) |
| CYLD | CAGTTATATTCTGTGATGTTTCAAGAGAACATCACAGAATATAACTG | (161) |
| | GAGGTGTTGGGGACAAAGGTTCAAGAGACCTTTGTCCCCAACACCTC | (162) |
| | GTGGGCTCATTGGCTGAAGTTCAAGAGACTTCAGCCAATGAGCCCAC | (163) |
| | GAGCTACTGAGGACAGAAATTCAAGAGATTTCTGTCCTCAGTAGCTC | (164) |
| UBIQUITIN | TCAGCAGGATGCTCAGGAGTTCAAGAGACTCCTGAGCATCCTGCTGA | (165) |
| CARBOXYL- | GAAGTTCTCCATCCAGAGGTTCAAGAGACCTCTGGATGGAGAACTTC | (166) |
| TERMINAL | GCCGGTCCCCACCAGCAGCTTCAAGAGAGCTGCTGGTGGGGACCGGC | (167) |
| HYDROLASE 2 | CACTCGGGAGTTGAGAGATTTCAAGAGAATCTCTCAACTCCCGAGTG | (168) |
| UBIQUITIN | GCCCTTGGGTCTGTTTGACTTCAAGAGAGTCAAACAGACCCAAGGGC | (169) |
| SPECIFIC | CTCAACACTAAACAGCAAGTTCAAGAGACTTGCTGTTTAGTGTTGAG | (170) |
| PROTEASE 3 | GATTTCATTGGACAGCATATTCAAGAGATATGCTGTCCAATGAAATC | (171) |
| (USP3) | CATGGGGCACCAACTAATTTTCAAGAGAAATTAGTTGGTGCCCCATG | (172) |
| UBIQUITIN | GGTGTCTCTGCGGGATTGTTTCAAGAGAACAATCCCGCAGAGACACC | (173) |
| CARBOXYL- | AGTTCAGTAGGTGTAGACTTTCAAGAGAAGTCTACACCTACTGAACT | (174) |
| TERMINAL | GAGTTCCTGAAGCTCCTCATTCAAGAGATGAGGAGCTTCAGGAACTC | (175) |
| HYDROLASE 23 | GGATTTGCTGGGGGCAAGGTTCAAGAGACCTTGCCCCCAGCAAATCC | (176) |
| UBP-32.7 | CTCAGAAAGCCAACATTCATTCAAGAGATGAATGTTGGCTTTCTGAG | (177) |
| | CGCATTGTAATAAGAAGGTTTCAAGAGAACCTTCTTATTACAATGCG | (178) |
| | GGGAGGAAAATGCAGAAATTTCAAGAGAATTTCTGCATTTTCCTCCC | (179) |
| | TTACAAATTTAGGAAATACTTCAAGAGAGTATTTCCTAAATTTGTAA | (180) |
| HOMO SAPIENS | GTTATGAATTGATATGCAGTTCAAGAGACTGCATATCAATTCATAAC | (181) |
| UBIQUITIN SPE- | GTGATAACACAACTAATGGTTCAAGAGACCATTAGTTGTGTTATCAC | (182) |
| CIFIC PROTEASE | GTAGAGGAGAGTTCTGAAATTCAAGAGATTTCAGAACTCTCCTCTAC | (183) |
| 13 (ISOPEP- | GCCTCTAATCCTGATAAGGTTCAAGAGACCTTATCAGGATTAGAGGC | (184) |
| TIDASE T-3) | | |
| UBIQUITIN | GATGATCTTCAGGCTGCCATTCAAGAGATGGCAGCCTGAAGATCATC | (185) |
| CARBOXYL- | GTATGGACAAGAGCGTTGGTTCAAGAGACCAACGCTCTTGTCCATAC | (186) |
| TERMINAL | CGAACCCTTCTGGAACAGTTTCAAGAGAACTGTTCCAGAAGGGTTCG | (187) |
| HYDROLASE 28 | GTGGCATGAAGATTATAGTTTCAAGAGAACTATAATCTTCATGCCAC | (188) |
| UBIQUITIN | GGTGAACAAGGACAGTATCTTCAAGAGAGATACTGTCCTTGTTCACC | (189) |
| CARBOXYL- | GCAATAGAGGATGATTCTGTTCAAGAGACAGAATCATCCTCTATTGC | (190) |
| TERMINAL | TCTGTGAATGCCAAAGTTCTTCAAGAGAGAACTTTGGCATTCACAGA | (191) |
| HYDROLASE 14 | CACACCAGGGAAGGTCTAGTTCAAGAGACTAGACCTTCCCTGGTGTG | (192) |
| DUB1 | GCAGGAAGATGCCCATGAATTCAAGAGATTCATGGGCATCTTCCTGC | (193) |
| | GAATGTGCAATATCCTGAGTTCAAGAGACTCAGGATATTGCACATTC | (194) |
| | TGGATGATGCCAAGGTCACTTCAAGAGAGTGACCTTGGCATCATCCA | (195) |
| | GCTCCGTGCTAAACCTCTCTTCAAGAGAGAGAGGTTTAGCACGGAGC | (196) |
| MOUSE USP27 | GCCTCCACCTCAACAGAGGTTCAAGAGACCTCTGTTGAGGTGGAGGC | (197) |
| HOMOLOG | CTGCATCATAGACCAAATCTTCAAGAGAGATTTGGTCTATGATGCAG | (198) |
| | GATCACTACATACATTTCCTTCAAGAGAGGAAATGTATGTAGTGATC | (199) |
| | GTAAAGAGAGCAGAATGAATTCAAGAGATTCATTCTGCTCTCTTTAC | (200) |
| UBIQUITIN | CGCGGGGCGCAGTGGTATCTTCAAGAGAGATACCACTGCGCCCCGCG | (201) |
| CARBOXYL- | CAGAAGGCAGTGGGGAAGATTCAAGAGATCTTCCCCACTGCCTTCTG | (202) |
| TERMINAL | GCCTGGGAGAATCACAGGTTTCAAGAGAACCTGTGATTCTCCCAGGC | (203) |
| HYDROLASE 4 | ACCAGACAAGGAAATACCCTTCAAGAGAGGGTATTTCCTTGTCTGGT | (204) |
| TRE-2 | CACATCCACCACATCGACCTTCAAGAGAGGTCGATGTGGTGGATGTG | (205) |
| | GTCACAACCCAAGACCATGTTCAAGAGACATGGTCTTGGGTTGTGAC | (206) |
| | CTCAACAGGACAAATCCCATTCAAGAGATGGGATTTGTCCTGTTGAG | (207) |
| | TAGATCAATTATTGTGGATTTCAAGAGAATCCACAATAATTGATCTA | (208) |
| UBIQUITIN CAR- | GGAACACCTTATTGATGAATTCAAGAGATTCATCAATAAGGTGTTCC | (209) |
| BOXYL-TERMINAL | CTTTAACAGAAATTGTCTCTTCAAGAGAGAGACAATTTCTGTTAAAG | (210) |
| HYDROLASE 15 | CCTATGCAGTACAAAGTGGTTCAAGAGACCACTTTGTACTGCATAGG | (211) |
| (UNPH-2). | GATCTTTTCTTGCTTTGGATTCAAGAGATCCAAAGCAAGAAAAGATC | (212) |
| KIAA1372 | CAGCATCCTTCAGGCCTTATTCAAGAGATAAGGCCTGAAGGATGCTG | (213) |
| | GATAGTGACTCGGATCTGCTTCAAGAGAGCAGATCCGAGTCACTATC | (214) |
| | GACATCACAGCCCGGGAGTTTCAAGAGAACTCCCGGGCTGTGATGTC | (215) |
| | GGACACAGCCTATGTGCTGTTCAAGAGACAGCACATAGGCTGTGTCC | (216) |
| BRCA1 | GTGGAGGAGATCTACGACCTTCAAGAGAGGTCGTAGATCTCCTCCAC | (217) |
| ASSOCIATED | CTCTTGTGCAACTCATGCCTTCAAGAGAGGCATGAGTTGCACAAGAG | (218) |
| PROTEIN-1 | ACAGGGCCCCTGCAGCCTCTTCAAGAGAGAGGCTGCAGGGGCCCTGT | (219) |
| | GAAGACCTGGCGGCAGGTGTTCAAGAGACACCTGCCGCCAGGTCTTC | (220) |

A preferred embodiment of the method (2) of the invention concerns the following steps:
1. Generation of the short hairpin DNA containing the antisense- and sense-strand of the coding region of a gene (e.g. firefly luciferase; p53). Antisense and sense-strand are separated by a spacer of 5 to 9 bp (?).
2. Generation of constructs for the expression of the above mentioned shRNA under the control of a constitutive or inducible promoter (Pol II or Pol III dependent).
3. Insertion of the mentioned expression constructs into a ubiquitous expressed Pol II dependent locus by homologous recombination in ES cells.
4. Analysis of the constitutive and inducible inhibition of gene expression (e.g. firefly luciferase; p53) in ES cells (e.g. through Western blot analysis).
5. Generation of mice using the mentioned ES cells and analysis of the inhibition of gene expression in several tissues (e.g. firefly luciferase; p53; e.g. through Western blot analysis).

The vector according to embodiment (4) of the invention is suitable for stable or transient integration. Said vector is suitable for gene transfer.

The technology of the present application provides for the following advantages:
(i) A stable and body wide inhibition of gene expression by generating transgenic animals (such as mice).
(ii) A reversible inhibition of gene expression using the inducible constructs.

The invention is furthermore described by the following examples which are, however, not to be construed so as to limit the invention.

### EXAMPLES

Methods

Plasmid Construction:

All plasmid constructs were generated by standard DNA cloning methods. The U6- and the H1-promoter fragments were amplified from human genomic DNA. The Renilla luciferase (Rluc) and firefly luciferase (Fluc) coding regions (Promega) were placed 3′ of the adenovirus splice acceptor site (Friedrich and Soriano, Genes Dev. 9:1513-23 (1991); see SEQ ID NOs: 3, 5 and 7) to facilitate transcription from the endogenous rosa26 promoter. For homologous recombination in embryonic stem cells, the expression cassettes were flanked by homology regions for the Rosa26 locus (see SEQ ID NO: 2).

Cell Culture:

Culture and targeted mutagenesis of F1 (C57BL/6×129Sv/Ev) ES cells were carried out as previously described (Hogan, B., Beddington, R., Costantini, F. and Lacy, E. (1994). A Laboratory Manual. In Manipulating the Mouse Embryo. (Cold Spring Harbor Laboratory Press, Cold Spring Harbor N.Y.), pp. 253-289.). For Cre-mediated deletion, ES cells were transiently transfected with 5 μg of the cre expression plasmid pCAGGScre (SEQ ID NO: 18). 24 h after transfection, 1000 ES cells were plated on a 10 cm dish. Individual clones were isolated and analyzed for cre-mediated recombination by Southern blot using standard protocols.

For induction, cells were cultivated in cell culture medium containing 1 μg/ml Doxycycline (Sigma) for 2 days.

For transient transfection of cells, mixtures of 350 ng supercoiled plasmid DNA pre-complexed with the FuGene6 transfection reagent (Roche Diagnostics GmbH, Mannheim, Germany) were added to the cells in 250 μl medium, according to the manufacturers protocol. Each plasmid mixture contained 50 ng of the beta-galactosidase expression vector CMV-lacZ (SEQ ID NO: 17), 100 ng of CAGGS-Fluc (SEQ ID NO: 15), 100 ng of the tet inducible H1shRNA vector (SEQ ID NO: 11, 12 or 13), and pUC19 to a total amount of 350 ng DNA. For each sample to be tested four individual wells were transfected. One day after the addition of the DNA preparations, the cell culture medium was exchanged.

Luciferase Measurement:

Stably transfected cells: 25.000 cells were plated on a 24 well plate and analyzed 48 h later. Luciferase activity was detected using the Dual-Glotm Luciferase Assay System (Promega) and a Lumat LB 9507 (EG&G Berthold) according to the manufacturers protocols.

Transiently transfected cells: The cells were lysed 48 hours after transfection with 100 μl/well of lysate reagent supplemented with protease inhibitors (β-galactosidase reporter gene assay kit, Roche Diagnostics). The lysates were centrifuged and 20 μl were used to determine the μ-galactosidase activities using the β-galactosidase reporter gene assay (Roche Diagnostics) according to the manufacturers protocol in a Lumat LB 9507 luminometer (Berthold). To measure Luciferase activity, 20 μl of lysate was diluted into 250 μl assay buffer (50 mM glycylglycin, 5 mM MgCl₂, 5 mM ATP) and the relative light units (RLU) were counted in a Lumat LB 9507 luminometer after addition of 100 μl of a 1 mM Luciferin (Roche Diagnostics) solution.

Generation of Chimeric Mice:

Recombinant ES cells were injected into blastocysts from Balb/C mice and chimeric mice were obtained upon transfer of blastocysts into pseudopregnant females using standard protocols (Hogan et al. Manipulating the Mouse Embryo: A Laboratory Manual. Cold Spring Harbor Laboratory Press, Cold Spring Harbor N.Y. 253-289 (1994)).

Luciferase Measurement in Organs:

Organs were homogenized at 4° C. in lysis buffer (0.1M KH₂PO₄, 1 mM DTT, 0.1% Triton® X-100) using a tissue grinder. Spin for 5′ at 2000×g (4° C.) to pellet debris and assay supernatant for Luc activities using the Dual Luciferase Assay (Promega, Inc.) according to the manufacturer protocol.

### Example 1

The firefly luciferase gene along with a splice acceptor sequence (Friedrich and Soriano, Genes Dev. 9:1513-23 (1991)) was inserted into the first allele of the rosa26 locus by homologous recombination in ES cells. The shRNA expression cassettes under the control of the H1 or the U6 promoter and a Renilla luciferase gene were inserted into the second allele of the rosa26 locus by homologous recombination. The Renilla luciferase gene was used as a reference to normalize the values of firefly luciferase activity.

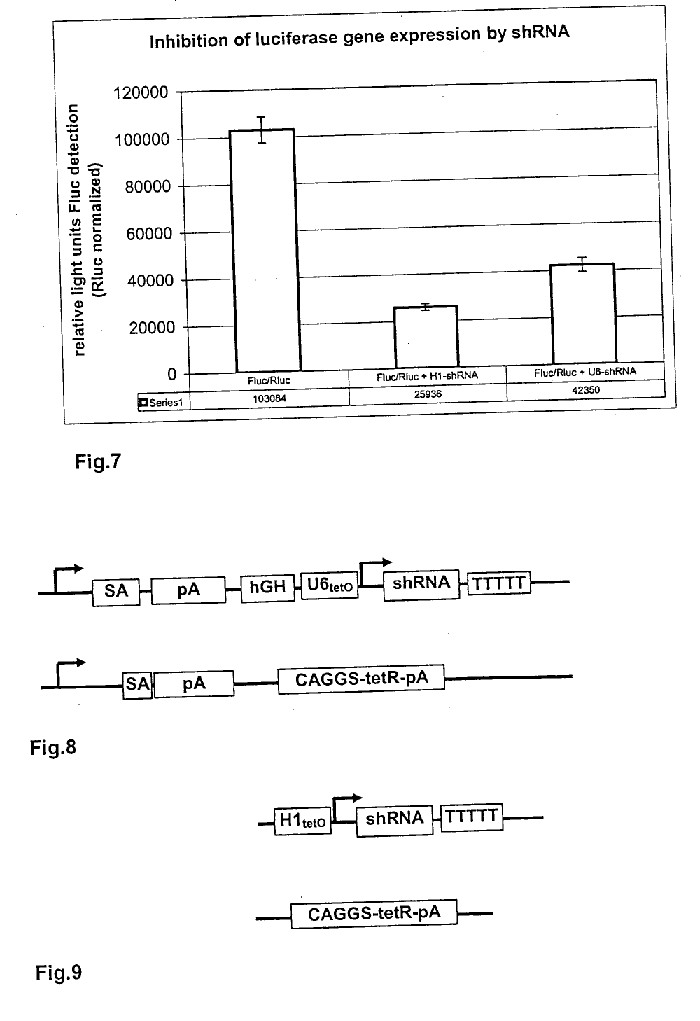

Deleting the loxP-flanked shRNA expression cassette by transient transfection of a cre expression plasmid generated the negative control. FIG. 7 shows the expression of the firefly luciferase in the absence and the presence of the shRNA expression cassette. Expression of the shRNA under the control of the H1 promoter resulted in a ˜75% reduction of firefly luciferase activity, whereas the repression mediated by the U6-shRNA construct was ˜60%.

### Example 2

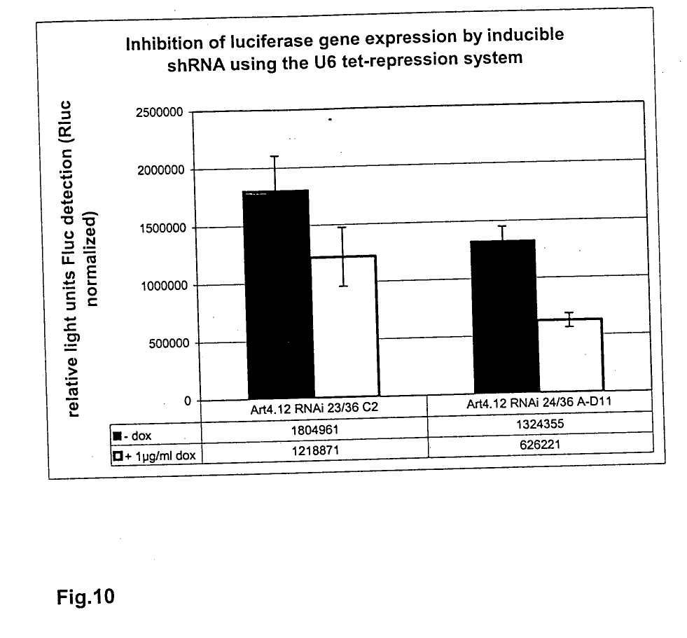

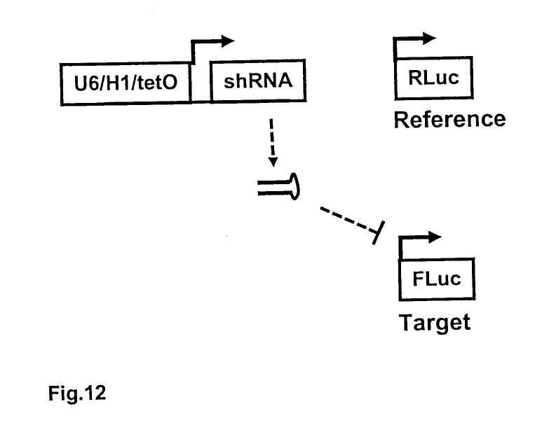

The shRNA expression cassette under the control of the U6 promoter containing tet-operator sequences, and a Renilla luciferase gene were inserted into the first allele of the rosa26 locus (FIGS. 8 and 12; SEQ ID NOs:9 and 10). The firefly luciferase gene under the control of the endogenous rosa26 promoter and a tet repressor expression cassette (Gossen and Bujard, PNAS. 89: 5547-5551 (1992)) was inserted into the second allele of rosa26 by homologous recombination in ES cells (FIG. 8; SEQ ID NO:14). To determine the extent of shRNA-mediated gene silencing, the cells were treated for 2 days in the presence or in the absence of 1 μg/ml Doxycycline in the cell culture medium. FIG. 10 shows the firefly luciferase activity measured in the cell extracts. Doxycycline inducible expression of the shRNA under the control of the U6_{tetO1} promoter (SEQ ID NO:9) resulted in a ˜30% reduction of firefly luciferase activity, whereas repression mediated by the U6_{tetO2}-construct (SEQ ID NO 10) reached ˜50%.

### Example 3

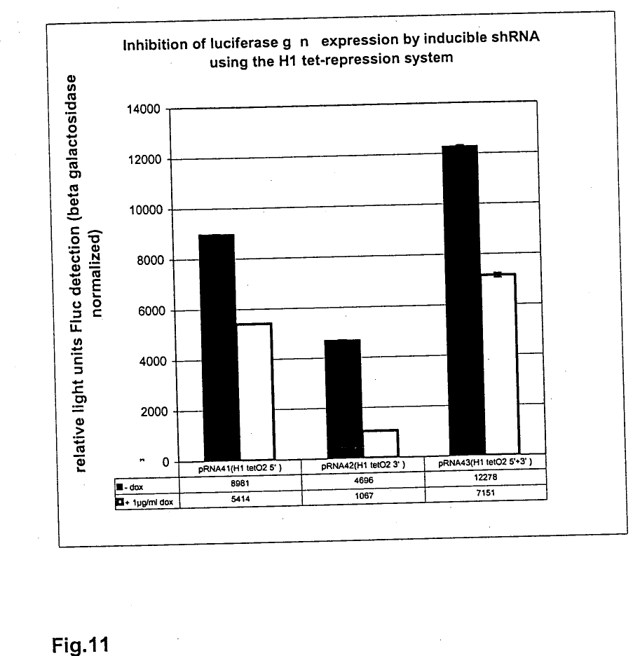

NIH3T3 cells were transiently transfected with constructs expressing the luciferase and tet repressor genes (SEQ ID NOs:15 and 16) together with the shRNA constructs under the control of the H1 promoter containing tet-operator sequences (sequence ID NOs:11, 12 and 13; FIG. 9). FIG. 11 shows the expression of firefly luciferase in the absence and in the presence of 1 μg/ml doxycycline. The highest degree of doxycycline inducible shRNA expression was achieved with the H1_{tetO2/3′}-promoter, resulting in a ˜80% reduction of firefly luciferase activity.

### Example 4

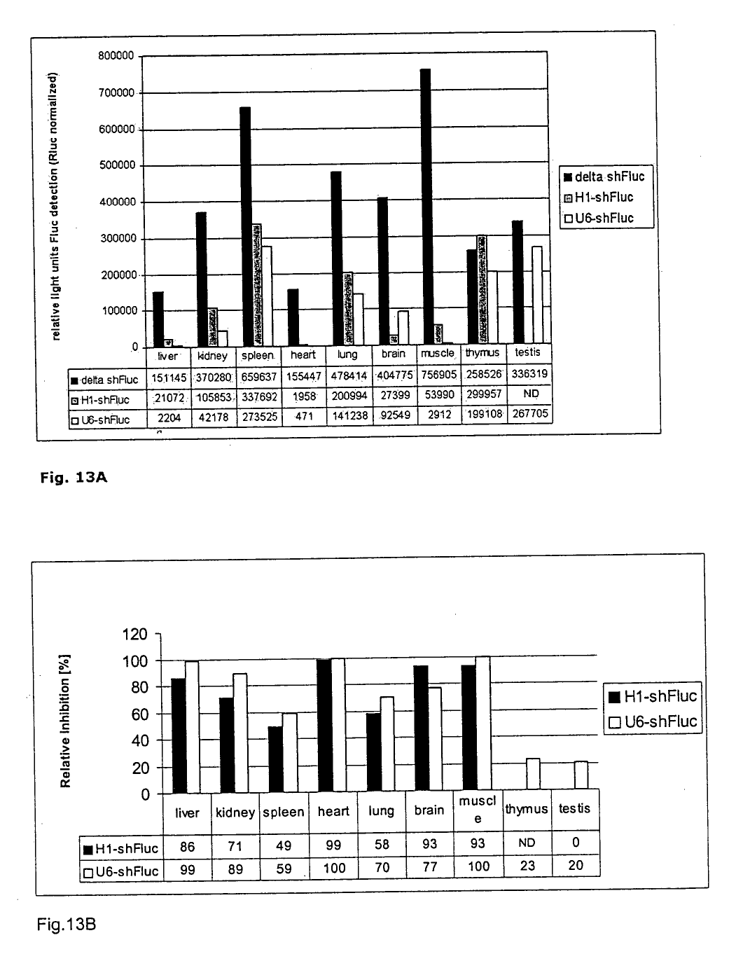

To examine the activity of the Rosa26/U6- and H1-shRNA transgenes in vivo, recombinant ES cells were injected into diploid blastocysts and chimeric mice were obtained upon transfer of blastocysts into pseudopregnant females. FIG. 13 shows the activity of firefly luciferase in different organs in the presence and absence of the shRNA expression cassettes. The Renilla luciferase gene at the second Rosa26 allele served as a reference to normalize the values of firefly luciferase activity. Expression of the shRNA under the control of both, the U6 as well as the H1 promoter resulted in efficient repression of firefly luciferase activity in most organs (FIG. 13A), with the highest degree of reduction (>90%) in liver, heart, brain and muscle (FIG. 13B).

1. A method for constitutive and/or inducible gene knock down in a vertebrate, or in a tissue culture or cells of a cell culture derived from a vertebrate, which comprises stably integrating an expression vector comprising a short hairpin RNA (shRNA) construct under control of a ubiquitous promoter into the genome of the vertebrate, of the tissue culture or of the cells of the cell culture.

2. The method of claim 1, wherein the expression vector is suitable for stabile integration into the genome of a vertebrate; or into the genome of the tissue culture or of cells of the cell culture.

3. The method of claim 1, wherein the expression vector contains homologous sequences suitable for integration at a defined genomic locus through homologous recombination in the genome of the vertebrate, in the genome of the tissue culture or in the genome of the cells of the cell culture including embryonic cells.

4. The method of claim 3, wherein the expression vector contains homologous sequences suitable for integration at a polymerase II dependent locus.

5. The method of claim 4, wherein the polymerase II dependent locus is selected from the group consisting of a Rosa26, collagen, RNA polymerase, actin and HPRT locus.

6. The method of claim 1, wherein the expression vector further contains functional sequences selected from the group consisting of splice acceptor sequences, polyadenylation sites, selectable marker sequences, etc.

7. The method of claim 1, wherein the ubiquitous promoter is selected from the group consisting of polymerase I, II and III dependent promoters.

8. The method of claim 7, wherein the ubiquitous promoter is a polymerase II or III dependent promoter.

9. The method of claim 7, wherein the ubiquitous promoter is selected from the group consisting of a CMV promoter, a CAGGS promoter, a snRNA promoter such as U6, a RNAse P RNA promoter such as H1, a tRNA promoter, a 7SL RNA promoter, a 5 S rRNA promoter, etc.

10. The method of claim 1, wherein the ubiquitous promoter is a constitutive promoter.

11. The method of claim 1, wherein the ubiquitous promoter is an inducible promoter.

12. The method of claim 11, wherein the inducible promoter is a promoter containing an operator sequence selected from the group consisting of tet, Gal4, lac, etc.

13. The method of claim 1, wherein said vertebrate is a non-human vertebrate.

14. The method of claim 13, wherein said vertebrate is a mouse or fish.

15. The method of claim 1, wherein the expression vector is a Pol III dependent promoter driven shRNA construct suitable to be integrated into a ubiquitously active Pol II dependent locus.

16. The method of claim 15, wherein the promoter is a constitutive H1 or U6 promoter.

17. The method of claim 15, wherein the promoter is an inducible U6 or H1 promoter.

18. The method of claim 1, wherein the expression vector is a Pol II dependent promoter driven shRNA construct suitable to be integrated into a ubiquitously active Pol II dependent locus.

19. The method of claim 18, wherein the promoter is an inducible CMV promoter.

20. The method of claim 1, wherein the shRNA comprises at least one DNA segment
A-B-C
wherein
A is a 15 to 35 bp DNA sequence with at least 95% complementarily to the gene to be knocked down;
B is a spacer DNA sequence having 5 to 9 bp forming the loop of the expressed RNA hair pin molecule, and
C is a 15 to 35 bp DNA sequence with at least 85% complementarily to the sequence A.

21. The method of claim 20, wherein A is a 19 to 29 bp DNA sequence.

22. The method of claim 20, wherein the DNA sequence A has 100% complementarily to the gene to be knocked down.

23. The method of claim 20, wherein C is a 19 to 29 bp DNA sequence.

24. The method of claim 1, wherein the shRNA comprises a stop and or polyadenylation sequence.

25. The method of claim 1, wherein the expression vector is integrated at a polymerase dependent locus of the living organism, tissue culture or cell culture.

26. The method of claim 1, wherein the method for constitutive and/or inducible gene knock down in a vertebrate comprises integrating the expression vector into ES cells of the vertebrate.

27. A vertebrate, or tissue or cell culture derived from a vertebrate having stably integrated, preferably at a polymerase II dependent locus of the vertebrate, tissue culture or cells of the cell culture, an expression vector comprising a short hairpin RNA (shRNA) construct under control of a ubiquitous promoter.

28. The vertebrate tissue or cell culture of claim 27, which is or is derived from a non-human vertebrate.

29. The vertebrate tissue or cell culture of claim 27, which is or is derived from a mouse or fish.

30. An expression vector comprising a short hairpin RNA (shRNA) construct under control of a ubiquitous promoter
